(19) United States
(12) Patent Application Publication
(10) Pub. No.: US 2005/0191246 A1
(43) Pub. Date: Sep. 1, 2005

(54) Powders comprising low molecular dextran and methods of producing those powders

(75) Inventors: Karoline Bechtold-Peters, Biberach-Rissegg (DE); Richard Fuhrherr, Neurnburg (DE); Wolfgang Friess, Iffeldorf (DE); Stefan Bassarab, Biberach (DE); Patrick Garidel, Nordersted (DE); Torsten Schultz-Fademrecht, Biberach-Stafflangen (DE)

Correspondence Address: MICHAEL P. MORRIS;BOEHRINGER INGELHEIM CORPORATION, 900 RIDGEBURY ROAD, P. O. BOX 368, RIDGEFIELD, CT, 06877-0368, US

(73) Assignee: Boehringer Ingelheim Pharma GmbH & Co. KG, Ingelheim (DE)

(21) Appl. No.: 10/999,471

(22) Filed: Nov. 30, 2004

Related U.S. Application Data
(60) Provisional application No. 60/532,094, filed on Dec. 23, 2003.

(30) Foreign Application Priority Data
Dec. 13, 2003 (DE) 103 58 387.4

(51) Int. Cl. A61K 39/395; A61K 38/43; A61L 9/04; A61K 9/14; A61K 38/18

(52) U.S. Cl. 424/46; 424/130.1; 514/12; 424/94.1; 514/59

(57) ABSTRACT

Disclosed are powders, preferably spray-dried powders, which contain a pharmaceutical active substance and low-molecular dextran as excipient. Also disclosed are processes for preparing such powders and methods of administering them by inhalation.

### APPLICATION DATA

This application claims benefit to German application DE 103 58 387.4 filed Dec. 13, 2003 and U.S. provisional application 60/532,094 filed Dec. 23, 2003.

### FIELD OF THE INVENTION

The invention relates to the use of low-molecular dextran (Mw: ≦10,000 Dalton) for the preparation and stabilisation of powders which contain a pharmaceutical active substance. The powders are preferably produced by spray-drying. The present invention also relates to powders, preferably spray-dried powders, which contain low-molecular dextran and a pharmaceutical active substance. The present invention relates particularly to protein- or peptide-containing powders and methods of producing them.

### BACKGROUND

Active substances/active substance preparations formulated in aqueous solutions are in some cases prone to instability which may lead to reduced bioactivity and increased incompatibilities. One possible method of stabilisation is offered for example by spray-drying, in which the pharmaceutical active substance is dried by spraying in a current of hot air. The pharmaceutical active substances are usually sprayed in the presence of excipients which on the one hand should maintain the stability of the active substances and on the other hand should improve the properties of the spray-dried powders.

A crucial factor in stabilising by spray-drying is the immobilisation of the active substance in an amorphous matrix. The amorphous state has high viscosity with low molecular mobility and low reactivity. The glass transition temperature of a spray-dried powder is an important parameter as it indicates the temperature range at which the transition from the stable, amorphous state into the less stable rubber-like state takes place. Advantageous excipients must be capable of forming an amorphous matrix with the highest possible glass transition temperature in which the active substance is embedded. Substances with a low glass transition temperature can flow even at low temperatures and lead to unstable powder formulations. The choice of excipients thus depends particularly on their stabilising qualities. In addition, however, factors such as the pharmaceutical acceptance of the excipients and its influence on particle formation, dispersibility and flow properties play a decisive role.

Spray-drying is a suitable process for increasing the chemical and physical stability of pharmaceutical active substances of the peptide/protein type (cf. Maa et al., 1998, Pharmaceutical Research, 15(5), 768-775). Particularly in the field of pulmonary treatment spray drying is used to produce peptide/protein-containing powdered medicaments (U.S. Pat. No. 5,626,874; U.S. Pat. No. 5,972,388; Broadhead et al., 1994, J. Pharm Pharmacol., 46(6), 458-467). The administration of peptide/proteins by inhalation is an alternative to traditional methods of administration in systemic diseases, as pharmaceutical products taken by inhalation may develop not only a local but also a systemic activity (WO 99/07340). The prerequisite for this is that the average particle size is in the range from 1-10 μm, preferably 1-7.5 μm, so that the particles can penetrate deep into the lungs and thus enter the bloodstream. DE-A-179 22 07, for example, describes the preparation of corresponding spray dried particles which are sufficiently dispersible for medical application (inhalation). In the meantime a number of methods of producing inhalable particles have been described (WO 95/31479; WO 96/09814; WO 96/32096; WO 96/32149; WO 97/41833; WO 97/44013; WO 98/16205; WO 98/31346; WO 99/66903; WO 00/10541; WO 01/13893; Maa et al., 1998, supra; Vidgren et al., 1987, Int. J. Pharmaceutics, 35,139-144; Niven et al., 1994, Pharmaceutical Research, 11(8), 1101-1109).

Sugar and alcohols thereof such as, for example, trehalose, lactose, saccharose or mannitol and various polymers have proved suitable as excipients (Maa et al., 1997, Pharm. Development and Technology, 2(3), 213-223; Maa et al., 1998, supra; Dissertation Adler, 1998, University of Erlangen; Costantino, et al., 1998, J. of Pharm. Sciences, 87(11), 1406-1411).

However, the excipients predominantly used have various drawbacks. The addition of trehalose and mannitol, for example, impairs the flow properties of spray-drying formulations (C. Bosquillon et al., 2001 Journal of Controlled Release, 70(3), 329-339). Moreover, mannitol has a tendency to recrystallise in amounts of more than 20 percent by weight (Costantino et al., 1998, supra), as a result of which its stabilising effects are dramatically reduced. Lactose, a frequently used excipient, does improve the flow properties of spray-drying formulations (C. Bosquillon et al., 2001, supra), but is problematic particularly in the formulation of peptide/protein-containing active substances, as lactose can enter into destabilising Maillard reactions with peptides/proteins as a result of its reducing property.

Dextrans with a molecular weight of 40 to 512 kDa are predominantly used in the freeze-drying of peptide/protein-containing active substances. They are amorphous by nature with a high glass transition at the same time. These high-molecular dextrans are only capable of entering into adequate hydrogen bridge bonds with peptides/proteins to a limited extent because of their rigid skeleton and thus ensure adequate stabilisation during freeze-drying. To compensate for this disadvantage they are sometimes combined with disaccharides (Allison et al., 2000, J. Pharm. Sci., 89(2),199-214). A further disadvantage of high-molecular dextrans resides in their high allergenic potential (dextran anaphylaxis).

One aim of the invention was to provide new excipients for the production of powdered pharmaceutical preparations. The corresponding powdered preparations should be characterised, among other things, by good stability on storage and, where possible, by being inhalable.

A further aim of the present invention was to provide new excipients for the preparation of spray-dried pharmaceutical preparations. The corresponding powdered pharmaceutical preparations should again be characterised by good long-term stability and, where possible, by being inhalable.

A further aim of the present invention was to provide new excipients for the preparation of peptide/protein-containing pharmaceutical formulations, particularly for those produced by spray-drying. The corresponding peptide/protein-containing pharmaceutical preparations should again be characterised by good long-term stability and, where possible, by being inhalable.

Another aim of the present invention was to provide pharmaceutical preparations for administration by inhalation, either in the form of a dry powder or a propellant-containing metered dose aerosol or a propellant-free inhalant solution.

The objectives on which the invention is based are achieved by the embodiments described below and by the objects/methods recited in the claims.

### SUMMARY OF THE INVENTION

The present invention relates to powders, preferably spray-dried powders, which contain a pharmaceutical active substance and low-molecular dextran with a molecular weight between about 500 and 10,000 Dalton (Da), preferably between about 500 and 5,000 Da, and particularly preferably between about 500 and 1,500 Da. Surprisingly, it was found that the corresponding powders after being spray-dried i) form an amorphous structure, ii) result in a relatively high yield (of at least 75% based on the solid used), iii) have a very high glass transition temperature (up to 65° C.) and iv) have a low tendency to recrystallisation. As another important advantage over e.g. spray-dried trehalose corresponding spray-dried powders which contain low-molecular dextran have improved flow properties. Another advantage over the powdered pharmaceutical preparations described in the prior art, particularly over known powdered spray-dried pharmaceutical preparations, resides in the particularly advantageous process and storage stability of the dextran-containing powders according to the invention described herein.

The amount of low-molecular dextran is preferably in relation to the dry mass of the powder between 50 and 99.99% % by weight (w/w), and according to another preferred embodiment between 55 and 99.99% (w/w), preferably between 60 and 99.99% (w/w).

The pharmaceutically active substance is preferably a biological macromolecule which may be a polypeptide or a protein, e.g. a growth factor, enzyme or antibody. The invention therefore relates in particular to spray-dried powders containing (a) a proportion of 50 to 99.99%, preferably 60 to 99.99% (w/w) of low-molecular dextran (relative to the dry mass of the powder) and (b) a biological macromolecule as pharmaceutical active substance, preferably in a concentration between 0.01 and 40% (w/w), again relative to the dry mass of the powder, the sum of the percentages by weight of low-molecular dextran and biological macromolecule being at most 100% (w/w).

The spray-dried powders according to the invention may contain in addition to the low-molecular dextran other excipients, such as for example amino acids, peptides, proteins or sugars. Particularly advantageous are powders which contain in addition to the stabilising low-molecular dextran and the pharmaceutical active substance at least one amino acid, a dipeptide, a tripeptide and/or a salt. According to a preferred embodiment the present invention relates to spray-dried powders which contain relative to their dry mass (a) between 60 and 98.99% (w/w) of a low-molecular dextran, (b) between 1 and 20% (w/w) of at least one amino acid and/or at least one peptide as a further excipient and (c) at least 0.01% (w/w) of a pharmaceutical active substance. Preferably the further excipient is the amino acid isoleucine or a di- or tripeptide containing at least one isoleucine group. According to a special embodiment the present invention relates to spray-dried powders which contain in relation to their dry mass (a) approximately 60 to 89.99% (w/w) of a low-molecular dextran, (b) approximately 10 to 20% (w/w) of an amino acid, preferably isoleucine and (c) approximately 0.01 to 30% (w/w) of a pharmaceutical active substance, preferably a peptide/protein, for example an antibody. According to another special embodiment the present invention relates to spray-dried powders which contain in relation to their dry mass (a) approximately 60 to 98.99% (w/w) of a low-molecular dextran, (b) approximately 1 to 20% (w/w) of an isoleucine-containing tripeptide, preferably triisoleucine and (c) approximately 0.01 to 39% (w/w) of a pharmaceutical active substance, preferably a peptide/protein, for example an antibody.

According to another embodiment the present invention relates particularly to spray-dried powders which contain low-molecular dextran and at least one pharmaceutical active substance, the spray-dried powder having a glass transition temperature of more than 40° C., preferably more than 45° C., more preferably more than 50° C., even more preferably more than 55° C. and particularly preferably more than 60° C. The amount of excipient added, particularly the amount of low-molecular dextran in the powder, is primarily responsible for the corresponding glass transition temperature.

According to another embodiment the present invention relates to pharmaceutical compositions for administration by inhalation, which contain one of the powders according to the invention described herein or consist of these powders. Preferred pharmaceutical compositions for this purpose are those which contain the powders according to the invention as propellant-containing metered dose aerosols or propellant-free inhalable solutions. The spray-dried powders according to the invention used to prepare the pharmaceutical composition are characterised according to another embodiment by a high proportion of inhalable particles with a mean aerodynamic particle diameter (MMAD) of less than 10 μm, preferably from 0.5-7.5 μm, more preferably from 0.5-5.5 μm, most preferably from 0.5-5.0 μm.

The invention also provides processes for preparing the corresponding spray-dried powders according to the invention, characterised in that a solution or suspension which contains at least one low-molecular dextran and a pharmaceutical active substance is produced and this is sprayed under suitable conditions. The temperature for the spraying process is preferably between 50 and 200° C. (inflow temperature) and 30 and 150° C. (outflow temperature).

### DESCRIPTION OF THE FIGURES

FIG. 1 shows the aggregate formation after spray-drying, forced storage and reconstitution. Aqueous solutions were sprayed, containing a) 10% (w/w) IgG content, b) 1% (w/w) IgG and 9% trehalose content and c) 1% (w/w) IgG and 9% dextran₁₀₀₀ content. The dextran-containing powders are characterised by a low content of aggregates.

FIG. 2 shows the aggregate formation after spray-drying, forced storage and reconstitution. Aqueous solutions were sprayed, containing a) 10% (w/w) IgG content, b) 1% (w/w) IgG, 1% (w/w) isoleucine and 8% trehalose content and c) 1% (w/w) IgG, 1% (w/w) isoleucine and 8% (w/w) dextran₁₀₀₀ content. The dextran-containing powders are characterised by a low content of aggregates.

FIG. 3 shows the Mass Mean Aerodynamic Diameter (MMAD) of different powders produced by spray-drying aqueous solutions containing dextran₁₀₀₀, isoleucine and IgG. The solutions were prepared as described under EXAMPLES and sprayed. All the powders have a MMAD of less than 7.5 μm. The diagram shows the influence of the isoleucine content on the MMAD with constant total solids concentrations and spray parameters. A higher isoleucine content in the formulation reduces the MMAD. Total Solid: proportion of solids in the spray solution. Cyclone II: Büchi Cyclone. Cyclone II: Büchi High-performance Cyclone.

FIG. 4 shows the Fine Particle Fraction (FPF) with a Cut Off Diameter of less than 5 μm for various powders which were prepared by spray-drying aqueous solutions containing dextran₁₀₀₀, isoleucine and IgG. The solutions were prepared and sprayed as described under EXAMPLES. All the powders have a FPF of more than 30%, or more than 35%. Total Solid: proportion of solids in the spray solution. Cyclone I: Büchi Cyclone. Cyclone II: Büchi High-performance Cyclone.

FIG. 5 shows the Mass Mean Aerodynamic Diameter (MMAD) of various powders which were prepared by spray-drying aqueous solutions containing dextran₁₀₀₀, triisoleucine and IgG. The solutions were prepared and sprayed as described under EXAMPLES. Both powders have a MMAD of less than 5 μm, or less than 4 μm. Total

Solid: proportion of solids in the spray solution. Büchi Cyclone. Cyclone II: Büchi High-performance Cyclone.

FIG. 6 shows the Fine Particle Fraction (FPF) with a Cut Off Diameter of less than 5 μm for various powders which were prepared by spray-drying aqueous solutions containing dextran₁₀₀₀, triisoleucine and IgG. The solutions were prepared and sprayed as described under EXAMPLES. Both the powders have a FPF of more than 55%, or 58%. Total Solid: proportion of solids in the spray solution. Büchi Cyclone. Cyclone II: Büchi High-performance Cyclone.

FIG. 7 shows the aggregate formation after spray-drying, up to one years storage at 2-8, 25 and 40° C. with subsequent reconstitution. An aqueous solution was sprayed, containing 1% (w/w) IgG, 1% (w/w) isoleucine and 8% (w/w) dextran₁₀₀₀ (see Example 2). The dextran-containing powder is characterised by a low content of aggregates after 3, 6, 9 and 12 months storage at 2-8° C., 25° C., 40° C.

FIG. 8 shows the aggregate formation after spray-drying, up to one years storage at 2-8, 25 and 40° C. with subsequent reconstitution. An aqueous solution was sprayed, containing 0.33% (w/w) IgG, 0.33% (w/w) isoleucine and 2.66% (w/w) dextran₁₀₀₀ (see Example 2). The dextran-containing powder is characterised by a low content of aggregates after one years storage at 2-8° C., 25° C., 40° C.

FIG. 9 shows the aggregate formation after spray-drying, up to one years storage at 2-8, 25 and 40° C. with subsequent reconstitution. An aqueous solution was sprayed, containing 0.33% (w/w) IgG, 0.33% (w/w) triisoleucine and 2.66% (w/w) dextran₁₀₀₀ (see Example 3). The dextran-containing powder is characterised by a low content of aggregates after one years storage at 2-8° C., 25° C., 40° C.

FIG. 10 shows the residual monomer content after spray-drying, forced storage and reconstitution. Aqueous solutions were sprayed, containing a) 3.33% (w/w) lysozyme, b) 0.33% (w/w) lysozyme and 3.0% dextran₁₀₀₀, c) 0.33% (w/w) lysozyme, 0.33% (w/w) isoleucine and 2.66% (w/w) dextran₁₀₀₀ and d) 0.33% (w/w) lysozyme, 0.33% (w/w) triisoleucine and 2.66% (w/w) dextran₁₀₀₀. The dextran-containing powder is characterised by a high residual monomer content.

FIG. 11 shows the aggregate content after spray-drying, forced storage and reconstitution. Aqueous solutions were sprayed, containing a) 3.33% (w/w) calcitonin, b) 0.33% (w/w) calcitonin and 3.0% dextran₁₀₀₀ and c) 0.33% (w/w) calcitonin, 0.33% (w/w) isoleucine and 2.66% (w/w) dextran₁₀₀₀. The dextran-containing powder is characterised by a low aggregate content.

FIG. 12 shows an inhaler for the administration of dry powdered preparations by inhalation.

### DETAILED DESCRIPTION OF THE INVENTION

Definitions

Terms and designations used within the scope of this specification have the following meanings defined below. The details of weight and percentages by weight are based on the dry mass of the powders or the solids content of the solutions/suspensions to be sprayed, unless stated otherwise.

The term “dextran 1” or “dextran₁₀₀₀” refers to a low-molecular dextran with a mean molecular weight of about 1.000 Dalton. The molecular weights given in this patent specification for dextran in each case relate to the mean molecular weight. This means that the dextrans used are generally polymorphous. The mean molecular weight indicates that at least 50%, preferably 60%, more preferably 70%, even more preferably 80%, even more preferably 90%, even more preferably 92%, even more preferably 94%, even more preferably 96%, even more preferably 98% and even more preferably 99% of the dextrans have a molecular weight corresponding to the numerical value.

The term “spray-dried powder formulation” or “dry powder formulation” refers to powder formulations which usually contain less than about 10% (w/w) residual moisture, preferably less than 7% (w/w) residual moisture, most preferably less than 3% (w/w) residual moisture and even more preferably less than 2% (w/w) residual moisture. The residual moisture is essentially dependent on the type and amount of the pharmaceutical active substance in the powder formulation.

The term “amorphous” means that the powdered formulation contains less than 10% crystalline fractions, preferably less than 7%, more preferably less than 5%, and most preferably less than 4, 3, 2, or 1%.

The word “inhalable” means that the powders are suitable for pulmonary administration. Inhalable powders can be dispersed and inhaled by means of an inhaler so that the particles enter the lungs and are able to develop a systemic activity optionally through the alveoli. Inhalable particles may have an average particle diameter, for example, of between 0.4-10 μm (MMD=mass median diameter), usually between 0.5-5 μm, preferably between 1-3 μm and/or an average aerodynamic particle diameter (MMAD=mass median aerodynamic diameter) of between 0.5-10 μm preferably between 0.5-7.5 μm, more preferably between 0.5-5.5 μm, even more preferably 1-5 μm and particularly preferably between 1-4.5 μm.

“Mass Median Diameter” or “MMD” is a measurement of the average particle size distribution as the powders according to the invention are generally polydispersed. The results are expressed as diameters of the total volume distribution at 50% total throughflow. The MMD values can be determined for example by laser diffractometry (cf.: Chapter EXAMPLES, Method), although of course any other conventional method may be used (e.g. electron microscopy, centrifugal sedimentation).

The term mean aerodynamic particle diameter (=mass median aerodynamic diameter (MMAD)) indicates the aerodynamic particle size at which 50% of the particles of the powder normally have a smaller aerodynamic diameter. In cases of doubt the reference method for determining the MMAD is the method specified in this patent specification (cf. the chapter EXAMPLES, Method).

The term “fine particle fraction” (FPF) describes the inhalable part of a powder consisting of particles with a particle size of ≦5 μm MMAD. In powder which is well dispersible the FPF is more than 20%, preferably more than 30%, more particularly more than 40%, and more preferably more than 50%, even more preferably more than 55%. The expression “Cut Off Diameter” used in this context indicates which particles are taken into account when determining the FPF. An FPF of 30% with a Cut Off Diameter of 5 μm (FPF 5) means that at least 30% of all the particles in the powder have a mean aerodynamic particle diameter of less than 5 μm.

The term “spray solution” means aqueous solutions or suspensions in which the pharmaceutical active substance together with at least one excipient is dissolved/suspended.

The term “time of flight” is the name of a standard method of measurements, as described in more detail in the Chapter EXAMPLES. In a time of flight measurement the MMAD and FPF are determined simultaneously (cf.: Chapter EXAMPLES, Method).

The terms “pharmaceutically acceptable excipients”, “carriers” or “matrices” refer to excipients which may optionally be contained in the formulation within the scope of the invention. The excipients may for example be administered to the lungs without having any significantly unfavourable toxicological effects on the subjects or on the subjects' lungs.

The term “pharmaceutically acceptable salts” includes for example the following salts, but is not restricted thereto: salts of inorganic acids such as chloride, sulphate, phosphate, diphosphate, bromide and nitrate salts. Also, salts of organic acids, such as malate, maleate, fumarate, tartrate, succinate, ethylsuccinate, citrate, acetate, lactate, methanesulphonate, benzoate, ascorbate, para-toluenesulphonate, palmoate, salicylate and stearate, and also estolate, gluceptate and lactobianate salts.

The term “pharmaceutically acceptable cations” includes, without being restricted thereto, for example, lithium, sodium, potassium, calcium, aluminium and ammonium (including substituted ammonium).

By a “pharmaceutical active substance” “is meant a substance, medicine, composition or combination thereof which has a pharmacological, usually positive effect on an organism, an organ and/or a cell if the active substance is brought into contact with the organism, organ or cell. When introduced into a patient the effect may be local or systemic.

The term “biological macromolecule” refers to peptides, proteins, fats, fatty acids or nucleic acids.

The term “peptide” or “polypeptide” refers to polymers of amino acids consisting of two to a hundred amino acid groups. The term “peptide” or “polypeptide” is used as a pseudonym and includes both homopeptides and heteropeptides, i.e. polymers of amino acids consisting of identical or different amino acid groups. Thus, a “dipeptide” is made up of two peptidically linked amino acids, a “tripeptide” of three peptidically linked amino acids. The term “protein” used here refers to polymers of amino acids with more than 100 amino acid groups.

The term “analogues” refers to peptides/proteins in which one or more amino acids have been substituted, eliminated (e.g. fragments), added (e.g. derivatives with a C- or N-terminal extension) or otherwise modified from the native (wild-type) sequence. It is also possible to derivatise the native protein, e.g. by means of sugars, polyethyleneglycol or the like. Analogues have a bioactivity of at least 10, 20, 30 or 40%, preferably at least 50, 60 or 70% and particularly preferably at least 80, 90, 95 100% or more than 100% bioactivity of the native, non-synthetic protein.

The term “amino acid” denotes compounds which contain at least one amino group and at least one carboxyl group. Although the amino group is usually in the a position to the carboxyl group any other arrangement in the molecule is also possible. The amino acid may also contain other functional groups such as e.g. amino, carboxamide, carboxyl, imidazole, thio groups and other groups. Amino acids of natural or synthetic origin, racemically or optically active (D- or L-) including various stereoisomeric ratios are used. For example, the term isoleucine covers both D-isoleucine, L-isoleucine, racemic isoleucine and various ratios of the two enantiomers.

The term “pure protein formulation” refers to spray-dried powders consisting of one or more proteins and optionally a suitable buffer (typically 0 to 15% (w/w) relative to the weight of the dry powder). The powder basically contains no other excipients, i.e. the content of any other excipients is less than 1% (w/w) relative to the weight of the dry powder.

A “surface-active” substance is capable of reducing the surface tension of the solution in which it is dissolved. The surface activity is measured for example by the tensiometer method according to Lecomte du Noüy (Bauer, Frömming, Fuhrer, 6th edition).

Powders According to the Invention

The present invention relates to the preparation of new excipients for stabilising pharmaceutical active substances/active substance preparations. Thanks to the present invention it is possible to prepare powdered active substance formulations which are characterised by particular stability, particularly by a low aggregate and high monomer content. Later in the specification these new and surprisingly superior stabilising active substances will be described and characterised in more detail.

Dextran is usually a high-molecular glucose polymer. It may be prepared for example by cultivating Leuconostoc Mesenteroides B512F in the presence of saccharose.

Native dextran can be obtained by partial acid hydrolysis after corresponding purification steps in desired molecular weight fractions. Dextran is a (1->6) linked α-D-glucan with side chains bound to the O-3 positions. The degree of branching is usually about 5%. The branches are usually 1-2 glucose units long. The dextrans normally used have mean molecular weights significantly above 10,000 Da. (usually 40,000, 70,000 or 512,000 Da). The dextran claimed in the Examples of the invention, on the other hand, has only an mean molecular weight of up to 10,000 Da, preferably up to 5,000 Da, most preferably up to 1,500 Da. Within the scope of the present invention it has been found that dextran with a mean molecular weight of approximately 1,000 Da. is particularly suitable as a stabiliser in the preparation of particulate powders.

Advantages of the pharmaceutical dextrans:
USP and Ph. Eur. monographed
free from Leuconostoc antigen
permitted for i.v. administration
fully biodegradable to carbon dioxide and water
stable at ambient temperature for 5 years

Special advantage of low-molecular dextran (−1,000 Dalton)
low antigenicity.

The present invention therefore relates to powders, preferably spray-dried powders, containing a pharmaceutical active substance and a low-molecular dextran with a molecular weight between about 500 and 10,000 Dalton (Da), preferably between about 500 and 5,000 Da, and particularly preferably between about 500 to 1,500 Da. According to a particularly preferred embodiment the present invention relates to powders, preferably spray-dried powders, which contain in addition to a pharmaceutical active substance dextran with a mean molecular weight of about 1,000 Da.

Powders which have proved particularly advantageous are those powders, preferably spray-dried powders, whose content of low-molecular dextran in relation to the dry mass of the powder is between 50 and 99.99% (w/w), preferably between 55 and 99.99% (w/w), more preferably between 60 and 99.99% (w/w), for example 50, 50.1, 50.2 50.3, . . . 50.7, 50.8, 50.9 etc.; 51, 52, 53, . . . 58, 59, 60 etc.; 61, 62, 63, . . . 68.69, 70 etc.; 71, 72, 73, . . . 78, 79, 80 etc.; 81, 82, 83, . . . 88, 89, 90 etc.; 91, 92, 93, . . . 98 etc, 99.1, 99.2, 99.3, . . . 99.8, . 99.9, etc.; 99.91, 99.92, 99.93, . . . 99.98, 99.99 (w/w). Overall, the amount of low-molecular dextran should be selected so that the spray-dried powder is at least partially amorphous, preferably totally amorphous. The amount of low-molecular dextran may also be reduced to less than 50% (w/w), provided that other stabilising excipients are added to the powder in suitable amounts. Examples of other stabilising excipients can be found elsewhere in this patent specification.

The amount of pharmaceutical active substance in the dry mass of the powder according to the invention is generally between 0.01 and 50% (w/w), preferably between 0.33 and 50% (w/w), more preferably between 0.33 and 45% (w/w), even more preferably between 0.33 and 40% (w/w). According to a another preferred embodiment the amount of pharmaceutical active substance in the solid content of the powder according to the invention is between 0.33 and 35% (w/w), preferably between 0.33 and 30% (w/w), more preferably between 0.33 and 25% (w/w) and even more preferably between 0.33 and 10% (w/w). The amount is thus for example 0.01, 0.02, 0.03 . . . 0.08, 0.09, etc.; 0.1, 0.2, 0.3, . . . 0.8, 0.9 etc.; 1, 2, 3, . . . 8, 9, 10 etc.; 11, 12, 13, . . . 18, 19, 20 etc.; 21, 22, 23, . . . 28, 29, 30 etc.; 31, 32, 33, . . . 38, 39, 40 etc.; 41, 42, 43, . . . 48, 49, etc; 49.1, 49.2, 49.3, . . . 49.8, 49.9, etc.; 49.91, 49.92, 49.93, . . . 49.98, 49.99 (w/w).

The invention therefore relates to powders with a ratio of low-molecular dextran to active substance of for example 50/50, 51/49, 52/48, 53/47, 54/46, 55/45, 56/44, 57/43, 58/42, 59/41, 60/40, 61/39, 62/38, 63/37, 64/36, 65/35, 66/34, 67/33, 68/32, 69/31, 70/30, 71/29, 72/28, 73/27, 74/26, 75/25, 76/24, 77/23, 78/22, 79/21, 80/20, 81/19, 82/18, 83/17, 84/16, 85/15, 86/14, 87/13, 88/12, 89/11, 90/10, 91/9, 92/8, 93/7, 94/6, 95/5, 96/4, 97/3, 98/2, 99/1, 99.1/0.9, 99.2/0.8, 99.3/0.7, 99,4/0.6, 99.5/0.5, 99.6/0.4, 99.66/0.33, 99.7/0.3, 99.8/0.2, 99.9/0.1, 99.99/0.01 (w/w). If the particular powder contains one or more additional excipients, either the amount of low-molecular dextran, the amount of pharmaceutical active substance or both amounts can be reduced accordingly, the amount of low-molecular dextran relative to the dry mass of the powder preferably having one of the values between 50 and 99.99% (w/w).

Pharmaceutical active substances for the purposes of the invention include, in addition to those covered by the general definition, antibiotics, anti-viral active substances, antiepileptics, pain-relievers (analgesics), anti-inflammatory active substances or bronchodilators. They also include active substances which act for example on the peripheral nervous system, on adrenergic receptors, cholinergic receptors, the skeletal muscles, the cardiovascular system, the smooth muscle, the blood circulatory system, on synaptic points, neuroeffector connecting points, the endocrine system, the immune system, the reproductive system, the skeletal system, the autacoid systems, the alimentary and excretory systems, the histamine system and the central nervous system. Suitable active substances also include for example hypnotics and sedatives, psychic energizers, tranquillisers, anti-convulsants, muscle relaxants, anti-Parkinsons active substances, pain relievers, anti-inflammatory active substances, muscle contractants, anti-microbial active substances, hormonal active substances such as for example contraceptives, sympathomimetics, diuretics, fat metabolism regulating active substances, anti-androgenic active substances, antiparasitics, neoplastics, antineoplastics and hypoglycaemics.

The term pharmaceutical active substance also includes, for example, active substances which act on the respiratory system, for example against one of the following complaints: asthma, chronic obstructive pulmonary diseases (COPD), emphysemic chronic bronchitis, bronchopulmonary dysplasia (BPD), neonatal Respiratory Distress Syndrome (RDS), bronchiolitis, croup, post-extubation stridor, pulmonary fibrosis, pneumonia or cystic fibrosis (CF).

Representative examples of bronchodilators include among others beta-agonists, anticholinergics or methylxanthine. Examples of anti-inflammatory active substances are steroids, cromolyn, nedocromil and leukotriene inhibitors. Examples of steroids include beclomethasone, betamethasone, biclomethasone, dexamethasone, triamcinolone, budesonide, butixocort, ciclesonide, fluticasone, flunisolide, icomethasone, mometasone, tixocortol and loteprednol. Other examples are budesonide, fluticasone propionate, beclomethasone dipropionate, fometerol and triamcinolone acetonide.

Examples of antimicrobially active substances are erythromycin, oleandomycin, troleandomycin, roxithromycin, clarithromycin, davercin, azithromycin, flurithromycin, dirithromycin, josamycin, spiromycin, midecamycin, leucomycin, miocamycin, rokitamycin, andazithromycin and swinolide A; fluoroquinolones, for example ciprofloxacin, ofloxacin, levofloxacin, trovafloxacin, alatrofloxacin, moxifloxicin, norfloxacin, eoxacin, grepafloxacin, gatifloxacin, lomefloxacin, sparfloxacin, temafloxacin, pefloxacin, amifloxacin, fleroxacin, tosufloxacin, prulifloxacin, irloxacin, pazufloxacin, clinafloxacin and sitafloxacin; aminoglycosides such as for example gentamicin, netilmicin, paramecin, tobramycin, amikacin, kanamycin, neomycin; streptomycin, vancomycin, teicoplanin, rampolanin, mideplanin, colistin, daptomycin, gramicidin, colistimethate; polymixins such as for example polymixin B, capreomycin, bacitracin, peneme, penicillins including penicillinase-sensitive active substances wie penicillin G, penicillin V, penicillinase-resistant active substances such as methicillin, oxacillin, cloxacillin, dicloxacillin, floxacillin, nafcillin; active substances against gram-negative bacteria such as ampicillin, amoxicillin, hetacillin, cillin and galampicillin; anti-pseudomonal penicillins such as carbenicillin, ticarcillin, azlocillin, meziocillin, andpiperacillin; cephalosporins such as cefpodoxime, cefprozil, ceftbuten, ceftizoxime, ceftriaxon, cephalothin, cephapirin, cephalexin, cephradrin, cefoxitin, cefamandol, cefazolin, cephaloridin, cefaclor cefadroxil, cephaloglycin, cefuroxim, ceforanid, cefotaxim, cefatrizin, cephacetril, cefepim, cefixim, cefonizid, cefoperazon, cefotetan, cefinetazol, ceftazidim, loracarbef and moxalactam; monobactams such as aztreonam; and carbapenems such as for example imipenem, meropenem, pentamidin isethionate, albuterol sulphate, lidocaine, metaproterenol sulphate, beclomethasone dipropionate, triamcinolone acetamide, budesonide acetonide, fluticasone, ipratropium bromide, flunisolide, cromolyn sodium, ergotamine tartrate and, where applicable, analogues, agonists, antagonists, inhibitors and pharmaceutically usable salt forms thereof and the like.

The pharmaceutical active substance is preferably a biological macromolecule according to another embodiment. In accordance with the definition provided above this is intended to include for example peptides, proteins, fats, fatty acids or nucleic acids.

Biopharmaceutically important proteins/polypeptides include e.g. antibodies, enzymes, growth factors, e.g. steroids, cytokines, lymphokines, adhesion molecules, receptors and the derivatives or fragments thereof, but are not restricted thereto. Generally, all polypeptides which act as agonists or antagonists and/or have therapeutic or diagnostic applications are of value.

Suitable peptides or proteins for the purposes of the invention include for example insulin, insulin-like growth factor, human growth hormone (hGH) and other growth factors, tissue plasminogen activator (tPA), erythropoietin (EPO), cytokines, e.g. interleukines (IL) such as IL-1, IL-2, IL-3, IL-4, IL-5, IL-6, IL-7, IL-8, IL-9, IL-10, IL-11, IL-12, IL-13, IL-14, IL-15, IL-16, IL-17, IL-18, interferon (IFN)-alpha, -beta, -gamma, -omega or -tau, tumour necrosis factor (TNF) such as TNF-alpha, -beta or -gamma, TRAIL, G-CSF, GM-CSF, M-CSF, MCP-1 and VEGF. Other examples are monoclonal, polyclonal, multispecific and single chain antibodies and fragments thereof such as for example Fab, Fab′, F(ab′)₂, Fc and Fc′ fragments, light (L) and heavy (H) immunoglobulin chains and the constant, variable or hypervariable regions thereof as well as Fv and Fd fragments (Chamov et al., 1999, Antibody Fusion proteins, Wiley-Liss Inc.). The antibodies may be of human or non-human origin.

These include for example the classes known in man: IgA, IgD, IgE, IgG and IgM, with their various subclasses, for example IgA1, IgA2 and IgG1, IgG2, IgG3 and IgG4. Humanised and chimeric antibodies are also possible. Of particular therapeutic importance and hence a subject of the present invention are powder formulations which [contain] antibodies against for example various surface antigens such as CD4, CD20 or CD44, various cytokines, for example IL2, IL4 or IL5. Other Examples are antibodies against specific classes of immunoglobulin (e.g. anti-IgE antibodies) or against viral proteins (e.g. anti-RSV, anti-CMV antibodies, etc.).

Fab fragments (fragment antigen binding=Fab) consist of the variable regions of both chains which are held together by the adjacent constant regions. Other antibody fragments are F(ab′)₂ fragments which can be produced by proteolytic digestion with pepsin. By gene cloning it is also possible to prepare shortened antibody fragments consisting of only the variable region of the heavy (VH) and light chain (VL). These are known as Fv fragments (fragment variable=fragment of the variable part). Such antibody fragments are also referred to as single chain Fv fragments (scFv). Examples of scFv antibodies are known and described, cf. for example Huston et al., 1988, Proc. Natl. Acad. Sci. USA, 16, 5879ff.

In past years various strategies have been developed for producing multimeric scFv derivatives, such as e.g. dia-, tri- and pentabodies. The term diabody is used in the art to denote a bivalent homodimeric scFv derivative. Shortening the peptide linker in the scFv molecule to 5 to 10 amino acids results in the formation of homodimers by superimposing VH/VL chains. The diabodies may additionally be stabilised by inserted disulphite bridges. Examples of diabodies can be found in the literature, e.g. in Perisic et al., 1994 (Structure, 2, 1217ff). The term minibody is used in the art to denote a bivalent homodimeric scFv derivative. It consists of a fusion protein which contains the CH3 region of an immunoglobulin, preferably IgG, most preferably IgG1, as dimerisation region. This connects the scFv fragments by means of a hinge region, also of IgG, and a linker region. Examples of such minibodies are described by Hu et al., 1996, Cancer Res., 56, 3055ff. The term triabody is used in the art to denote a trivalent homotrimeric scFv derivative (Kortt et al., 1997, Protein Engineering, 10, 423ff). The direct fusion of VH-VL without the use of a linker sequence leads to the formation of trimers.

The fragments known in the art as mini antibodies which have a bi-, tri- or tetravalent structure are also derivatives of scFv fragments. The multimerisation is achieved by means of di-, tri- or tetrameric coiled coil structures (Pack, P. et al., 1993, Biotechnology, 11, 1271ff; Lovejoy, B. et al., 1993, Science, 259, 1288ff; Pack, P. et al., 1995, J. Mol. Biol., 246, 28ff).

A particularly preferred embodiment of the invention relates to a protein from the class of antibodies, more precisely type 1 immunoglobulin G. This is a humanised monoclonal antibody, with 95% human and 5% murine antibody sequences. The antibody has a molecular weight of about 148 Kilodalton (kDa), consisting of two light and two heavy chains and a total of four disulphide bridges.

Particularly advantageous are powders which contain as active substance a peptide or protein or a combination of peptide/peptide, peptide/protein or protein/protein. The corresponding biological macromolecules may make up between 0.01 to 50% (w/w) of the dry mass of the powder. The amount is thus for example 0.01, 0.02, 0.03 . . . 0.08, 0.09, 0.1, 0.2, 0.3 . . . 0.8, 0.9 etc.; 1, 2, 3, . . . 8, 9, 10 etc.; 11, 12, 13, . . . 18, 19, 20 etc.; 21, 22, 23, . . . 28, 29, 30 etc.; 31, 32, 33, . . . 38, 39, 40 etc.; 41, 42, 43, . . . 48, 49, 49.1, 49.2, 49.3, . . . 49.8, .49.9 etc.; 49.91, 49.92, 49.93, . . . 49.98, 49.99 (w/w).

Particularly advantageous powders according to the invention are powders, preferably spray-dried powders, with a ratio of low-molecular dextran to peptide/protein of 50/50, 51/49, 52/48, 53/47, 54/46, 55/45, 56/44, 57/43, 58/42, 59/41, 60/40, 61/39, 62/38, 63/37, 64/36, 65/35, 66/34, 67/33, 68/32, 69/31, 70/30, 71/29, 72/28, 73/27, 74/26, 75/25, 76/24, 77/23, 78/22, 79/21, 80/20, 81/19, 82/18, 83/17, 84/16, 85/15, 86/14, 87/13, 88/12, 89/11, 90/10, 91/9, 92/8, 93/7, 94/6, 95/5, 96/4, 97/3, 98/2, 99/1, 99.1/0.9, 99.2/0.8, 99.3/0.7, 99.4/0.6, 99.5/0.5, 99.6/0.4, 99.66/0.33, 99.7/0.3, 99.8/0.2, 99.9/0.1, 99.99/0.01 (w/w). If the corresponding powder contains one or more additional excipients, either the amount of low-molecular dextran, the amount of pharmaceutical active substance, or both amounts can be reduced accordingly, the amount of low-molecular dextran preferably having one of the values between 50 and 99.99% (w/w).

If the powders according to the invention contain very small proteins/peptides with a molecular weight of <10 kDa, preferably <5 kDa, such as for example growth factors, for example cytokines, the amount is preferably between 0.1 to 10% (w/w), more preferably between 0.2 to 5% (w/w) of the total weight of the powder. Accordingly, powders are preferred wherein the amount of cytokines is 0.2, 0.3, 0.4 . . . 0.8, 0.9 etc.; 1, 2, 3, . . . etc; 4.1, 4.2, 4.3, . . . 4.8, 4.9 etc.; 4.91, 4.92, 4.93, . . . 4.98, 4.99 (w/w).

If on the other hand the pharmaceutical active substance is one or more antibodies or a derivative thereof (preferred embodiment), the proportion of active substance in the solid content of the powder is between 0.01 and 50% (w/w), preferably between 0.1 and 50% (w/w), more preferably between 0.33 and 50% (w/w), for example 0.1, 0.2, 0.3, 0.33, . . . 0.66, 0.7, 0.8, 0.9etc.; 1, 2, 3, . . . 8, 9, 10etc.; 11, 12, 13, . . . 18, 19, 20 etc.; 21, 22, 23, . . . 28, 29, 30 etc.; 31, 32, 33, . . . 38, 39, 40etc.; 41, 42, 43, . . . 48, 49, etc; 49.1, 49.2, 49.3, . . . 49.8, 49.9 etc.; 49.91, 49.92, 49.93, . . . 49.98, 49.99 (w/w).

According to a particular embodiment the proportion of antibodies in the solids content of the powder is between 10 and 50% (w/w), more preferably between 10 and 30% (w/w), even more preferably between 10 and 20% (w/w). The invention relates, particularly advantageously, to powders, preferably spray-dried powders, with a ratio of low-molecular dextran to antibody of 50/50, 51/49, 52/48, 53/47, 54/46, 55/45, 56/44, 57/43, 58/42, 59/41, 60/40, 61/39, 62/38, 63/37, 64/36, 65/35, 66/34, 67/33, 68/32, 69/31, 70/30, 71/29, 72/28, 73/27, 74/26, 75/25, 76/24, 77/23, 78/22, 79/21, 80/20, 81/19, 82/18, 83/17, 84/16, 85/15, 86/14, 87/13, 88/12, 89/11 or 90/10 (w/w).

According to another embodiment the present invention relates to powders, preferably spray-dried powders, characterised in that the dry mass of the spray-dried powder contains a) at least 50% (w/w), preferably between 55 and 99.99% (w/w), most preferably between 60 and 99.99% (w/w) of low-molecular dextran and b) up to 30% (w/w) of a biological macromolecule, the sum of the percentages by weight of low-molecular dextran and biological macromolecule being at most 100% (w/w). A skilled man is in a position to prepare such powders. Thus, the skilled man knows that he can add at most 0.01% (w/w) of a pharmaceutical active substance relative to the total solids content of the solution which is to be sprayed, if the amount of low-molecular dextran is to be 99.99% (w/w).

The powders according to the invention may also contain other excipients, such as for example amino acids, peptides, non-biological or biological polymers, and/or one or more sugars. Other excipients known in the art are for example lipids, fatty acids, fatty acid esters, steroids (e.g. cholesterol) or chelating agents (e.g. EDTA) as well as various cations (see above). Excipients with a high glass transition temperature, for example above 40° C., preferably above 45° C., or above 55° C., are particularly preferred. A list of suitable excipients can be found for example in Kippe (Eds.), “Handbook of Pharmaceutical Excipient“ ” 3rd Ed., 2000.

Suitable protein-containing excipients include for example albumin (human or recombinant in origin), gelatine, casein, haemoglobin and the like. The sugars are preferably mono-, di-, oligo- or polysaccharides or a combination thereof. Examples of monosaccharides are fructose, maltose, galactose, glucose, d-mannose, sorbose and the like. Suitable disaccharides for the purposes of the invention include for example, lactose, sucrose, trehalose, cellobiose, and the like. Polysaccharides which may be used include in particular raffinose, melecitose, dextrin, starch and the like. Sugar alcohols include in addition to mannitol, xylitol, maltitol, galactitol, arabinitol, adonitol, lactitol, sorbitol (glucitol), pyranosylsorbitol, inositol, myoinositol and the like as excipients. Suitable amino acids include for example alanine, glycine, arginine, histidine, glutamate, asparagine, cysteine, leucine, lysine, isoleucine, valine, tryptophan, methionine, phenylalanine, tyrosine, citrulline, L-aspartyl-L-phenylalanine-methylester (=aspartame), trimethylammonioacetate (=betaine) and the like. Preferably, amino acids are used which act as buffers (e.g. glycine or histidine) and/or as dispersing agents. These latter groups include in particular predominantly hydrophobic amino acids, such as e.g. leucine, valine, isoleucine, tryptophan, alanine, methionine, phenylalanine, tyrosine, histidine or proline. Within the scope of the present invention it has proved particularly advantageous to use isoleucine in addition to the low-molecular dextran, preferably in a concentration of 5 to 20% (w/w), most preferably from 10 to 20% (w/w), even more preferably from 12 to 20% (w/w). It is also particularly advantageous to use di-, tri-, oligo- or polypeptides as further excipients, which contain one or more of these predominantly hydrophobic amino acid groups. Suitable examples of tripeptides include for example one or more of the following tripeptides: Leu-Leu-Gly, Leu-Leu-Ala, Leu-Leu-Val, Leu-Leu-Leu, Leu-Leu-Met, Leu-Leu-Pro, Leu-Leu-Phe, Leu-Leu-Trp, Leu-Leu-Ser, Leu-Leu-Thr, Leu-Leu-Cys, Leu-Leu-Tyr, Leu-Leu-Asp, Leu-Leu-Glu, Leu-Leu-Lys, Leu-Leu-Arg, Leu-Leu-His, Leu-Gly-Leu, Leu-Ala-Leu, Leu-Val-Leu, Leu-Met-Leu, Leu-Pro-Leu, Leu-Phe-Leu, Leu-Trp-Leu, Leu-Ser-Leu, Leu-Thr-Leu, Leu-Cys-Leu, Leu-Try-Leu, Leu-Asp-Leu, Leu-Glu-Leu, Leu-Lys-Leu, Leu-Arg-Leu and Leu-His-Leu. It has proved particularly advantageous to use tripeptides of general formulae: Ile-X-X; X-Ile-X; X-X-Ile, where X may be one of the following amino acids: alanine, glycine, arginine, histidine, glutamic acid, glutamine, asparagine, aspartic acid, cysteine, leucine, lysine, isoleucine (Ile), valine, tryptophan, methionine, phenylalanine, proline, serine, threonine, tyrosine, L-aspartyl-L-phenylalanine-methylester (=aspartame), trimethylammonio-acetate. Particularly preferred are corresponding tripeptides of formula (Ile)₂-X, for example Ile-Ile-X, Ile-X-Ile, or X-Ile-Ile, where X may again denote one of the amino acids listed above. These include for example the tripeptides: Ile-Ile-Gly, Ile-Ile-Ala, Ile-Ile-Val, Ile-Ile-Ile, Ile-Ile-Met, Ile-Ile-Pro, Ile-Ile-Phe, ile-Ile-Trp, Ile-Ile-Ser, Ile-Ile-Thr, Ile-Ile-Cys, Ile-Ile-Tyr, Ile-Ile-Asp, ile-Ile-Glu, Ile-Ile-Lys, ile-Ile-Arg, Ile-Ile-His, Ile-Gly-Ile, Ile-Ala-Ile, Ile-Val-Ile, Ile-Met-Ile, le-Pro-Ile, Ile-Phe-Ile, ile-Trp-Ile, Ile-Ser-Ile, Ile-Thr-Ile, Ile-Cys-Ile, Ile-Try-Ile, Ile-Asp-Ile, Ile-Glu-Ile, Ile-Lys-Ile, Ile-Arg-Ile, Ile-His-Ile. It is particularly advantageous to use Ile-Ile-Ile.

Suitable polymers include for example the polyvinylpyrrolidones mentioned above as excipients, derivatised celluloses, such as hydroxymethyl, hydroxyethyl or hydroxypropylethyl cellulose, polymeric sugars such as Ficoll, starch such as hydroxyethyl or hydroxypropyl starch, dextrins such as cyclodextrins (2-hydroxypropyl-β-cyclodextrin, sulphobutylether-β-cyclodextrin), polyethylenes, glycols and/or pectins.

The salts may be for example inorganic salts such as chlorides, sulphates, phosphates, diphosphates, hydrobromides and/or nitrate salts. Moreover the powders according to the invention may also contain organic salts, such as e.g. malates, maleates, fumarates, tartrates, succinates, ethylsuccinates, citrates, acetates, lactates, methanesulphonates, benzoates, ascorbates, paratoluenesulphonates, palmoates, salicylates, stearates, estolates, gluceptates or labionate salts. At the same time corresponding salts may contain pharmaceutically acceptable cations, such as for example sodium, potassium, calcium, aluminium, lithium or ammonium. It is particularly preferred to use corresponding cations in conjunction with the stabilisation of proteins. Consequently, according to another embodiment the present invention also relates to powders, preferably spray-dried powders, which contain a pharmaceutically acceptable salt in addition to the low-molecular dextran and the pharmaceutical active substance.

The present invention thus also relates to spray-dried powders which contain one or more pharmaceutically acceptable excipients and/or one or more salts in addition to the low-molecular dextran and the pharmaceutical active substance. The excipients may be, for example, the above-mentioned amino acids, peptides and their salts, sugars, polyols, salts of organic acids and/or polymers.

According to another embodiment the present invention relates to powders, preferably spray-dried powders, which contain in addition to the low-molecular dextran and the pharmaceutical active substance one or more amino acid(s), preferably one amino acid, as a further excipient. In this context the present invention also relates to powders which contain in relation to their dry mass at least 50% (w/w), preferably between 55 and 98.99% (w/w), most preferably between 60 and 98.99% (w/w) of low-molecular dextran, and between 1 and 20% (w/w) of amino acids and between 0.01 and 49% (w/w) of a pharmaceutical active substance, preferably a biological macromolecule, while the sum of the amounts by weight may be up to at most 100% (w/w). According to a preferred embodiment the amount of low-molecular dextran is at least 60% (w/w), preferably between 70 and 89.99% (w/w) in relation to the dry mass of the powder. In a corresponding formulation the amount of amino acids is preferably between 10 and 20% (w/w) and the amount of the pharmaceutical active substance is between 0.01 to 10% (w/w).

Consequently, according to another embodiment the present invention also relates to powders which contain or consist of, for example, 80% (w/w) of low-molecular dextran/19% (w/w) amino acid/1% (w/w) pharmaceutical active substance (80/19/1); (80/18/2); (80/17/3); (80/16/4); (80/15/5); (80/14/6); (80/13/7); (80/12/8); (80/11/9); (80/10/10); (70/19/11); (70/18/12); (70/17/13); (70/16/14); (70/15/15); (70/14/16); (70/13/17); (70/12/18); (70/11/19); (70/10/20); (60/20/20); (60/19/21); (60/18/22); (60/17/23); (60/16/24); (60/15/25); (60/14/26); (60/13/27); (60/12/28); (60/11/29); (60/10/30) or (70/20/10). If the proportion of active substance is reduced from 20% (w/w) to 0.01% (w/w), for example to 9.99, . . . 9.9, 9.8, 9.7 . . . 9.3, 9.2, 9.1 . . . 9, 8 7, 6, 5, 4, 3, 2, 1, . . . 0.9, 08, 0.7, . . . 0.66, . . . 0.6, 0.5, 0.4, 0.3, 0.2, 0.1, 0.09, 0.08, 0.07, 0.06, 0.05, 0.04, 0.03, 0.02, 0.01, while the proportion of amino acid remains constant, the amount of low-molecular dextran may be reduced accordingly to, for example, 80.01, . . . 80.1, 80.2, 80.3 . . . 80.8, 80.9, 81, 82, 83, 84, 85, 86, 87, 88, 89, . . . , 89.1, 89.2, 89.3, . . . 89.33, . . . 89.4, 89.5, 89.6, 89.7, 89.8, 89.9, . . . 89.91, 89.92, 89.93, . . . 89.97, 89.98, 89.99 (w/w), so that the sum of the amounts by weight of the individual powder ingredients in relation to the dry mass of the powder is 100% (w/w). By adding other excipients or salts the amount of low-molecular dextran, amino acids/peptides and/or pharmaceutical active substance can be adjusted/reduced accordingly, so that the parts by weight of the individual ingredients add up to a total of 100% (w/w).

If the amino acid added is isoleucine, according to another embodiment the powders according to the invention contain an amount of a) low-molecular dextran of at least 50% (w/w), preferably 55 to 89.99% (w/w), most preferably 60 to 89.99% (w/w), b) a proportion of 5 to 20% (w/w) isoleucine and c) at least 0.01% (w/w), preferably 0.01 to at most 45% (w/w) of a pharmaceutical active substance, preferably a peptide/protein, according to the invention. Preferably the amount of isoleucine is 10 to 20% (w/w), more preferably 12 to 20% (w/w) of the total solids content of the powder. Here again, the sum of the % by weight of the individual ingredients does not exceed 100% (w/w). The invention also relates to powders of the following composition: 85% (w/w) of low-molecular dextran/5% amino acid or peptide/10% (w/w) of pharmaceutical active substance (85/5/10), (84/6/10), (83/7/10), (82/8/10), (81/9/10), (80/10/10); (79/11/10); (78/12/10); (77/13/10); (76/14/10); (75/15/10); (74/16/10); (73/17/10); (72/18/10); (71/19/10); (70/20/10), while the amount of the pharmaceutical active substance may also be reduced from 10 to 0.01% (w/w), for example to 9.99, . . . 9.9, 9.8, 9.7 . . . 9.3, 9.2, 9.1 . . . 9, 8 7, 6, 5, 4, 3, 2, 1, . . . 0.9, 08, 0.7, . . . 0.66, . . . 0.6, 0.5, 0.4, 0.3, 0.2, 0.1, 0.09, 0.08, 0.07, 0.06, 0.05, 0.04, 0.03 0.02, 0.01 and accordingly the amount of low-molecular dextran may be increased to for example 80.01, . . . 80.1, 80.2, 80.3 . . . 80.8, 80.9, 81, 82, 83, 84, 85, 86, 87, 88, 89, . . . , 89.1, 89.2, 89.3, . . . 89.33, . . . , 89.4, 89.5, 89.6, 89.7, 89.8, 89.9, . . . 89.91, 89.92, 89.93, . . . , 89.97, 89.98, 89.99 (w/w), so that the sum of the parts by weight in relation to the dry mass of the powder makes up 100% (w/w). Therefore, the invention also relates to powders having the following composition: 80% (w/w) of low-molecular dextran/19% (w/w) of isoleucine/1% (w/w) of pharmaceutical active substance (80/19/1); (80/18/2); (80/17/3); (80/16/4); (80/15/5); (80/14/6); (80/13/7); (80/12/8); (80/11/9); (80/10/10); (70/19/11); (70/18/12); (70/17/13); (70/16/14); (70/15/15); (70/14/16); (70/13/17); (70/12/18); (70/11/19); (70/10/20); (60/19/21); (60/18/22); (60/17/23); (60/16/24); (60/15/25); (60/14/26); (60/13/27); (60/12/28); (60/11/29); (60/10/30). If other excipients or salts are added the amount of low-molecular dextran, isoleucine and/or pharmaceutical active substance should be adjusted accordingly so that the amounts by weight of the individual ingredients add up to 100% (w/w).

Another embodiment of the present invention relates to the use of low-molecular dextran and tripeptides for stabilising powders containing a pharmaceutical active substance, preferably a peptide, protein, or a mixture thereof. The present specification mentions by way of example some tripeptides which may be used together with low-molecular dextran to prepare the powders according to the invention. According to a particular embodiment the tripeptides are those which contain at least one isoleucine, preferably two isoleucines, or according to a particularly advantageous embodiment, consist of three isoleucines.

In connection with this, the invention relates to powders containing a) an amount of low-molecular dextran of at least 50% (w/w), preferably from 55 to 98.99% (w/w), most preferably from 60 to 98.99% (w/w), b) a proportion of 1 to 20% (w/w) of a tripeptide, preferably triisoleucine and c) 0.01 to at most 49% (w/w) of a pharmaceutical active substance, preferably a peptide/protein. Here again, the sum of the individual solids cannot add up to more than 100% (w/w). The invention also relates to powders of the following composition: 89% (w/w) of low-molecular dextran/1% tripeptide, preferably an isoleucine-containing tripeptide, most preferably triisoleucine/10% (w/w) of pharmaceutical active substance (89/1/10); (88/2/10); (87/3/10); (86/4/10); (85/5/10); (84/6/10); (83/7/10); (82/8/10); (81/9/10); (80/10/10); (79/11/10); (78/12/10); (77/13/10); (76/14/10); (75/15/10); (74/16/10);, (73/17/10); (72/18/10) or (71/19/10), while the amount of pharmaceutical active substance can also be reduced from 10 to 0.01% (w/w), for example to 9.99, . . . 9.9, 9.8, 9.7 . . . 9.3, 9.2, 9.1 . . . 9, 8 7, 6, 5, 4, 3, 2, 1, . . . 0.9, 08, 0.7, . . . 0.66, . . . 0.6, 0.5, 0.4, 0.3, 0.2, 0.1, 0.09, 0.08, 0.07, 0.06, 0.05, 0.04, 0.03 0.02, 0.01% (w/w) and accordingly the amount of low-molecular dextran may increase to for example 80.01, . . . 80.1, 80.2, 80.3 . . . 80.8, 80.9, 81, 82, 83, 84, 85, 86, 87, 88, 89, . . . 89.1, 89.2, 89.3, . . . 89.33, . . . 89.4, 89.5, 89.6, 89.7, 89.8, 89.9, . . . 89.91, 89.92, 89.93, . . . 89.97, 89.98, 89.99% (w/w), so that the sum of the amounts by weight in relation to the dry mass of the powder comes to 100% (w/w). Therefore the invention also relates to powders of the following composition: 80% (w/w) of low-molecular dextran/19% (w/w) tripeptide, preferably triisoleucine/1% (w/w) of pharmaceutical active substance (80/19/1); (80/18/2); (80/17/3); (80/16/4); (80/15/5); (80/14/6); (80/13/7); (80/12/8); (80/11/9); (80/10/10); (70/19/11); (70/18/12); (70/17/13); (70/16/14); (70/15/15); (70/14/16); (70/13/17); (70/12/18); (70/11/19); (70/10/20); (60/19/21); (60/18/22); (60/17/23); (60/16/24); (60/15/25); (60/14/26); (60/13/27); (60/12/28); (60/11/29); (60/10/30), while the amount of tripeptide, preferably triisoleucine can also be reduced from 10 to 1% (w/w), for example to 9.99, . . . 9.9, 9.8, 9.7 . . . 9.3, 9.2, 9.1 . . . 9, 8 7, 6, 5, 4, 3, 2.1.9, 1.8, 1.7, . . . 1.66, . . . 1.6, 1.5, 1.4, 1.3, 1.2, 1.1, 1% (w/w) and accordingly the amount of pharmaceutical active substance, preferably peptide/protein may be increased to for example 30.1, 30.2, 30.3 . . . 30.8, 30.9, 31, 32, 33, 34, 35, 36, 37, 38, 38.1, 38.2, 38.3, . . . 38.33, . . . , 38.4, 38.5, 38.6, 38.7, 38.8, 38.9, . . . 39 (w/w), so that the sum of the amounts by weight in relation to the dry mass of the powder comes to 100% (w/w). When the amount of tripeptide is reduced from 10 to 1 (w/w), as shown here, the proportion of low-molecular dextran in the powder can also be increased. When the proportion of active substance remains constant at for example 10% (w/w) powders can be produced with a dextran content of 80.1, 80.2, 80.3 . . . 80.8, 80.9, 81, 82, 83, 84, 85, 86, 87, 88, 88.1, 88.2, 88.3, . . . 88.33, . . . , 88.4, 88.5, 88.6, 88.7, 88.8, 88.9 or 89 (w/w).

According to another embodiment according to the invention the powders may additionally contain surfactants such as Tween 20, 40, 60, 80, Brij 35, Pluronic F 88 and Pluronic F 127 contain. These are preferably used in a concentration of 0.01-0.1% (w/w). Particularly preferred is a spray-dried powder which contains as excipient low-molecular dextran and additionally Tween 20, preferably in a concentration of 0.01-0.1% (w/w), as surfactant.

According to another embodiment the present invention also relates to pharmaceutical compositions containing one of the spray-dried powders described above.

Preparation of the Powder According to the Invention:

The present invention also provides processes for preparing one of the spray-dried powders described above. The process is characterised in that a solution/suspension to be sprayed, containing a pharmaceutical active substance and low-molecular dextran is sprayed below a temperature of 200/120° C. (inflow/outflow temperature) preferably at 150-185/70-95° C. The process according to the invention is described more fully by means of some Examples in the “EXAMPLES” section.

Basically, the powders according to the invention may be prepared by dissolving the pharmaceutical active substance, preferably a biological macromolecule in the form of a peptide or protein, in an aqueous solution, depending on the solubility conditions of the active substance in question. Usually, buffered solutions with a pH of 3-11, preferably 3.5-9 are used. When preparing inhalable powders an aqueous solution with a pH of 4-7.8 is particularly advantageous. In order to ensure sufficient solubility, the pH of the solution should be below the pI of the peptide/protein. The aqueous solution may optionally contain additional water-soluble organic solvents, such as e.g. acetone, alcohols or the like. Lower alcohols such as e.g. methanol, ethanol, propanol, (n or iso-propanol) or the like are particularly suitable. Mixed solvent systems of this kind normally contain between 10-20% (v/v) of a water-soluble organic solvent. The solid content in the solution to be sprayed is usually between 0.01-20% (w/w), preferably between 0.05-10% (w/w), particularly preferably between 0.1-5% (w/w). Within the scope of the present invention spray-dried powders were prepared starting from an aqueous solution with a solid content of 10% (w/w) or 3.33% (w/wt. %).

Usually, the excipient or a mixture of suitable excipients, as described above by way of example, is dissolved in a second container in highly pure water or a suitable buffer solution with a pH of 3 to 11, preferably 3.5 to 9 and particularly preferably 4.0 to 7.8 and mixed with the active substance solution in a second step. Then the solution/suspension is adjusted to the desired solid content with pure water or a suitable buffer solution with a pH of 3 to 11, preferably 3.5 to 9 and particularly preferably 4.0 to 7.8.

Consequently the present invention relates to a process for preparing a spray-dried powder, characterised in that
a) a pharmaceutical active substance is dissolved in an aqueous solution/suspension;
b) low-molecular dextran is dissolved in an aqueous solution/suspension;
c) if active substance and low-molecular dextran are dissolved in different solutions/suspension, these are mixed together;
d) the solution/suspension containing low-molecular dextran and the pharmaceutical active substance is sprayed below a temperature of 200/120° C., preferably 175/95° C.

The excipient content of low-molecular dextran in the solution/suspension which is to be sprayed is between 50% and 99.99% (w/w), preferably between 55% and 99.99% (w/w), most preferably between 60 and 99.99% (w/w) in relation to the solids content of the spray solution. The active substance concentration is normally between 0.01 and 50% (w/w), preferably between 0.01 and 40% (w/w), most preferably between 0.01 and 30% (w/w) in relation to the solids content of the solution or suspension which is to be sprayed. Starting from the powder compositions according to the invention described above, the skilled man is capable of preparing solutions/suspensions for spraying which result in the corresponding powder compositions after spraying.

Consequently the present invention also relates to processes for preparing a spray-dried powder, as described above, characterised in that the solids content of the solution/suspension which is to be sprayed contains between 50 and 99.99% (w/w), preferably between 60 and 99.99% (w/w) of low-molecular dextran. According to another preferred embodiment the present invention relates to a corresponding process characterised in that the solids content of the solution/suspension which is to be sprayed contains between 0.01 and 50% (w/w), preferably between 0.01 and 30% (w/w), most preferably between 0.33 and 30% (w/w) of a pharmaceutical active substance.

According to another embodiment of the present process a spray solution/suspension with a solids content of a) at least 50% (w/w), for example between 60 to 99.99% (w/w) of a low-molecular dextran and b) at least 0.01% (w/w), preferably 0.01 to 50% (w/w) of a pharmaceutical active substance, preferably a biological macromolecule, is prepared and sprayed, the sum of the % by weight being at most 100% (w/w). According to a preferred embodiment a spray solution/suspension with a solids content a) of low-molecular dextran of at least 60% (w/w), preferably between 60 to 99.99% (w/w), and b) 0.01 to 40% (w/w) of a pharmaceutical active substance, preferably a biological macromolecule, is prepared and sprayed, the sum of the % by weight of the solution or suspension being at most 100% (w/w).

Corresponding to the powders according to the invention described above, according to another embodiment the solution/suspension to be sprayed additionally contains one or more pharmaceutically acceptable excipients and/or one or more salts. The excipients are preferably amino acids, peptides or their salts, sugars, polyols, salts of organic acids and/or polymers.

Preferably the spray solution contains in addition to the pharmaceutical active substance and the low-molecular dextran one or more amino acids and/or peptides or proteins as other excipients. Consequently the present invention also relates to a process for preparing spray-dried powders characterised in that the solution/suspension to be sprayed contains, in relation to its solids content, a) at least 50% (w/w), preferably at least 60% (w/w) of low-molecular dextran, b) between 1 and 20% (w/w) of at least one amino acid and/or at least one peptide. Examples of suitable excipients including pharmaceutically acceptable salts, peptides and amino acids can be found under the heading “Powders according to the invention” in this specification.

According to another preferred embodiment the spray solution also contains in addition to low-molecular dextran one or more amino acids as a further excipient. Spray solutions/suspensions the solids content of which contains a) at least 50% (w/w), preferably 60 to 98.99% (w/w) of low-molecular dextran, b) 1 to 20% (w/w) amino acids, c) and at least 0.01% (w/w) of a pharmaceutical active substance, preferably a peptide/protein, such as for example an antibody, are advantageous. The amount of pharmaceutical active substance is preferably 0.01 to at most 30% (w/w) while the sum of the solids components is at most 100% (w/w). Anyone skilled in the art is capable of preparing corresponding powders and adapting the amounts by weight so that the sum of the solids components does not exceed 100% (w/w). If the amount (relative to the total solids content) of pharmaceutical active substance is for example 30% (w/w) and the amount of low-molecular dextran is 60% (w/w) the skilled man knows that he can add at most 10% (w/w) of amino acids to the spray solution/suspension.

According to another preferred embodiment the spray solution also contains isoleucine as a further excipient in addition to low-molecular dextran. Spray solutions/suspensions the solids content of which contains a) at least 50% (w/w), preferably 60 to 89.99% (w/w) of low-molecular dextran, b) 10 to 20% (w/w) of isoleucine, c) and at least 0.01% (w/w) of a pharmaceutical active substance, preferably a peptide/protein, such as for example an antibody, are advantageous. The amount of pharmaceutical active substance is preferably 0.01 to at most 30% (w/w) while the sum of the solids components is at most 100% (w/w). Anyone skilled in the art is capable of preparing corresponding powders and adapting the amounts by weight so that the sum of the solids components does not exceed 100% (w/w). If the amount (relative to the total solids content) of pharmaceutical active substance is for example 30% (w/w) and the amount of low-molecular dextran is 60% (w/w) the skilled man knows that he can add at most 10% (w/w) of isoleucine to the spray solution/suspension.

According to another embodiment the solution to be sprayed contains in addition to low-molecular dextran one or more tripeptides, preferably isoleucin-containing tripeptides, most preferably triisoleucine. Solutions or suspensions for spraying, the solids content of which contains a) at least 50% (w/w), preferably 60 to 98.99% (w/w) of low-molecular dextran, b) 1 to 19% (w/w) of a tripeptide, preferably triisoleucine, and c) at least 0.01% (w/w) of a pharmaceutical active substance, preferably a peptide/protein such as for example an antibody, are advantageous, while the sum of the solids components is at most 100% (w/w). The amount of pharmaceutical active substance is preferably 0.01 to at most 39% (w/w). Anyone skilled in the art is capable of preparing corresponding powders and adapting the amounts by weight so that the sum of the solids components does not exceed 100% (w/w). If the amount (relative to the total solids content) of pharmaceutical active substance is for example 30% (w/w) and the amount of low-molecular dextran is 60% (w/w) the skilled man knows that he can add at most 10% (w/w) of tripeptide, preferably triisoleucine, to the solution or suspension which is to be sprayed.

As mentioned previously, it is advantageous to prepare and spray solutions which are to be sprayed with a pH of between 3 and 11, preferably 3.5 and 9, most preferably between 4.0 and 7.8. Suitable buffer systems are known to the skilled man. Usually, it is particularly advantageous to use inorganic or organic salts as the buffer system.

Typically, the optimum excipient and protein content for each protein or peptide is determined experimentally. Preferred formulations of the invention may also contain at least one other excipient, in order to improve powder characteristics such as dispersibility and flow properties while retaining superior aggregation-inhibiting properties.

The spraying is done in conventional spray driers, for example in apparatus made by Messrs Niro A/S (Soeborg, DK), BOchi Labortechnik GmbH (Flawil, CH) or the like. The optimum conditions for the spray drying depend in each case on the corresponding formulation and should be determined experimentally. The gas used is typically air, but inert gases such as nitrogen or argon are also suitable. In addition, the spray drying temperature, i.e. the inlet temperature and outlet temperature, is determined in accordance with the temperature sensitivity of the active substance used, in each case depending on the stabilisers used. An inlet temperature of 50-200° C. is usual, while the outlet temperature is usually 30-150° C. Within the scope of the present invention an inlet temperature of approximately 170-185° C. and an outlet temperature of 80-100° C. was used. However, rather higher temperatures are also possible, for example an inlet temperature of up to 200° C., preferably 90-185° C. and an outlet temperature of up to 120° C., preferably 90-105° C., depending on the amount of stabiliser. Spraying is generally carried out at a pressure of approximately 20-150 psi, preferably at about 30 or 40-100 psi, for example at about 30, 40, 50, 60, 70, 80, 90 or 100 psi.

With regard to the Büchi sprayer the “Liquid Feed Rate” is normally between 0.1 and 100 ml/min, preferably between 0.1 and 30 ml/min, for example about 3 ml/min. In connection with this an Aspirator Flow Rate of 20-40 m³/h, preferably 30-40 m³/h, such as for example 35 m³/h and an atomising flow rate of 0.3-2.5 m³/h, preferably about 0.67 m³/h, has proved particularly suitable.

The spray-dried active substance formulations, preferably the powdered protein formulations, may optionally be subjected to a second gently drying (after-drying). The aim is to achieve a uniform residual water content in the formulations of less than 2% (w/w), and thereby improve both the active substance stability and also improve powder qualities such as the glass transition temperature, flowability and dispersibility. The conditions of the after-drying process must be selected such that the aggregate formation of the active substances is not significantly increased. This applies particularly to the use of biological macromolecules, such as for example the use of peptides/proteins. The spray-dried powdered active substance formulations are preferably prepared, processed and stored under dry conditions (at low relative humidity). The process of after-drying makes it possible to prepare and decant the powders at relatively high humidity levels. Surprisingly, the excipients to which the invention relates stabilise the proteins in the preferred formulations even under non-optimal processing and storage conditions.

Properties of the Spray-Dried Dry Powder Formulations

The dry powdered protein formulations prepared within the scope of this invention have a residual water content of less than 15% (w/w), usually less than 10% (w/w), and preferably less than 6% (w/w). More preferably the spray-dried powdered protein formulations have a residual water content of less than 3% (w/w), most preferably less than 2% (w/w) and most preferably between 0.2 and 2.0% (w/w). Formulations with a low residual moisture content generally exhibit improved stability during unpacking and storage. Moreover, the dry powdered protein formulations according to the invention are predominantly hygroscopic, i.e. they have a tendency to absorb moisture from their environment. To avoid this, powders of this kind are usually packaged in containers such as blister packs with the exclusion of moisture.

The stabilising effects of the excipients described here are capable of protecting the protein from extreme stresses during spray-drying and storage. In the absence of excipients spray-dried pure protein formulations form aggregates to a considerable degree. Process-related factors such as heat, shear stress and denaturing at the air/water interfaces cause aggregation (up to about 3.7% aggregates) during the spray-drying and subsequent after-drying (up to about 4.0% aggregates). During storage massive aggregate formation takes place (from about 11.8 to about 18.9% aggregates) as a result of the absence of the stabilising hydrate coat of the proteins.

The preferred spray-dried formulations of the invention, unlike the pure protein formulations, are capable of reducing the formation of aggregates both after spray-drying and also keeping it at a very low level under different storage conditions. As a result of spray-drying only about 1.1 to about 1.4% aggregates are formed in the preferred formulations, as against about 4.0% aggregates in pure protein formulations. Preferred formulations which are subjected to a second gentle drying, show no tendency to increased aggregate formation. Under particularly challenging storage conditions (40° C., 75% relative humidity) the preferred formulations (aggregates of −5.1 to ˜10.1%) are clearly superior to pure protein formulations (about 18.9% aggregates) and an analogous reference formulation with trehalose as excipient.

Formulations which have a significant stabilising effect on the incorporated proteins even during relatively short storage under particularly destabilising conditions (1 week at 40° C., 75% relative humidity) also stabilise proteins for long periods under far gentler standard storage conditions (e.g. 1 year, in the dry, at about 25° C.).

By varying the spray-drying conditions it is possible to produce powders which preferably have a mean particle size (MMD) of less than 20 μm, preferably less than 10 μm. According to a particularly preferred embodiment these particles according to the invention have a mean particle size of less than 7.5 μm, preferably less than 5 μm. Particularly preferred are particles with a mean particle size of less than 4 μm and more preferably less than 3.5 μm. Generally, it is also possible to prepare particles with a mean particle diameter of 0.1-5 μm, preferably 0.2-4 μm. In another embodiment non-respirable particles, e.g. lactose, with a particle size of at least 40 μm, preferably between 40 and 200 μm, are mixed with the corresponding powders.

Apart from the mean particle size (MMD) the inhalability essentially depends on the mean aerodynamic particle diameter (MMAD). The particles according to the invention preferably have an MMAD of less than 10 μm and more preferably less than 7.5 μm. Particularly advantageous are powders consisting of particles with an MMAD of less than 5.5 μm, preferably less than 5 μm, even more preferably less than 4.5 μm. The powders described in the Examples can be produced with corresponding particle sizes by a combination of optimum spray-drying conditions and the choice and concentration of excipients according to the invention. In particular the addition of amino acids and/or tripeptides leads to an improved particle performance with an increased proportion of inhalable particles with an MMAD of less than 7.5, preferably less than 5.5. By the addition of isoleucine or triisoleucine, inhalable powders with an FPF of more than 30%, preferably more than 40, more preferably more than 50 and even more preferably more than 55% can be prepared (see EXAMPLES).

The powders according to the invention are also characterised by a glass transition temperature of at least 45° C., preferably at least 50° C., more preferably at least 55° C., even more preferably at least 60° C. Particularly preferred powders have a glass transition temperature of at least 65° C. In general, the glass transition temperature of the dextran-containing powders according to the invention is 60 to 65° C. Accordingly, the present invention also relates to powders, preferably spray-dried powders, containing a pharmaceutical active substance and low-molecular dextran, wherein the glass transition temperature is 45° C. and more, preferably between 45 and 70° C. According to another preferred embodiment the glass transition temperature is 55° C. or above, preferably between 55 and 70° C.

Use of the Spray-Dried Powder

The powders according to the invention are suitable for the preparation of a pharmaceutical composition, preferably for preparing a medicament for inhalation.

Administration of the Powders According to the Invention

Basically, the powder preparations according to the invention may be administered directly as dry powders using so-called dry powder inhalers, or after reconstitution in the form of aerosols using so-called nebulisers. The inhalable powders according to the invention may be administered using inhalers known from the prior art.

Inhalable powders according to the invention may be administered, for example, by means of inhalers which deliver a single dose from a supply using a measuring chamber as described in U.S. Pat. No. 4,570,630A, or by other means as described in DE 36 25 685 A. Preferably, the inhalable powders according to the invention are packed into capsules (to produce so-called inhalettes) which are used in inhalers as described, for example, in WO 94/28958.

Other examples of suitable inhalers may be found inter alia in U.S. Pat. No. 5,458,135; U.S. Pat. No. 5,785,049 or WO 01/00263. Other suitable inhalers are known from WO 97/41031; U.S. Pat. No. 3,906,950 and U.S. Pat. No. 4,013,075. Other dispersion inhalers for dry powder preparations are described in EP 129 985; EP 472 598; EP 467 172 and U.S. Pat. No. 5,522,385.

The inhalable powders according to the invention may for example be administered using the inhaler known by the name Turbuhaler® (AstraZeneca LP) or with inhalers as disclosed for example in EP 237 507 A. Other suitable inhalers are the Rotahaler® or the Discus® (both made by GlaxoSmithKline Corp.), the Spiros™ inhaler (Dura Pharmaceuticals) and the Spinhaler® (Fiscon).

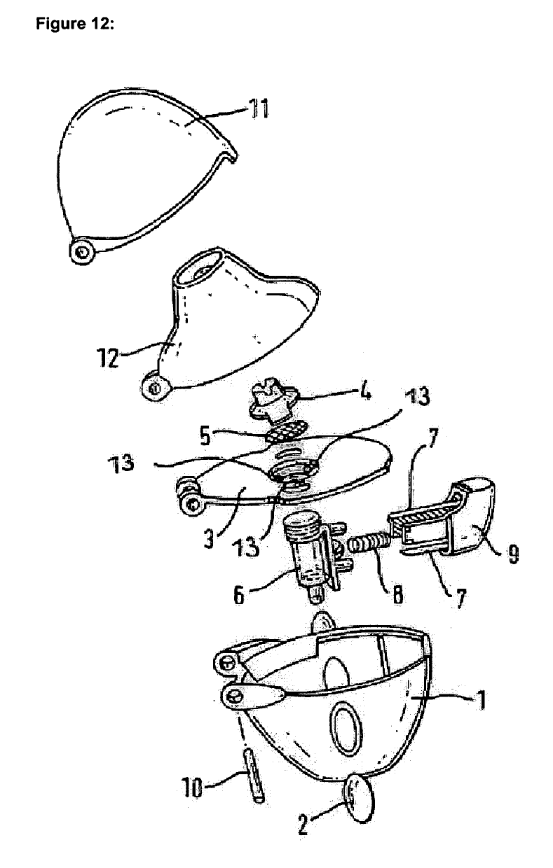

A particularly preferred inhaler for administering the pharmaceutical combination in inhalettes according to the invention is shown in FIG. 12. This inhaler (Handyhaler) for inhaling powdered pharmaceutical compositions from capsules is characterised by a housing 1 containing two windows 2, a deck 3 in which there are air inlet ports and which is provided with a screen 5 secured via a screen housing 4, an inhalation chamber 6 connected to the deck 3 on which there is a push button 9 provided with two sharpened pins 7 and movable counter to a spring 8, and a mouthpiece 12 which is connected to the housing 1, the deck 3 and a cover 11 via a spindle 10 to enable it to be flipped open or shut, as well as air through-holes 13 for adjusting the flow resistance.

If the inhalable powders according to the invention are to be packed into capsules (inhalettes) for the preferred use described above, the quantities packed into each capsule should be 1 to 30 mg.

The powders according to the invention may also be administered as propellant-containing inhalable aerosols. For this, the powders according to the invention are reconstituted in an aqueous solution. Suitable solutions are known in the art. For example, it is advantageous to reconstitute the powders in physiological solutions with a pH of 3-11, preferably 4-9. Reconstitution in an aqueous solution with a pH of 5.5-7.8 is particularly advantageous. The solution for reconstituting the powders according to the invention may also contain further excipients in the form of stabilisers, emulsifiers, surfactants or water-soluble organic solvents. Corresponding substances are known to the skilled man and described for example in Bauer, Lehrbuch der Pharmazeutischen Technologie, Wissenschaftl. Verlagsgesellschaft mbH, Stuttgart, 178-184; Adler, 1998, Journal of Pharmaceutical Sciences, 88(2), 199-208.

Corresponding inhalable aerosols which are prepared by reconstituting the powders according to the invention are also a subject of the present invention.

The propellant gases which may be used to prepare the inhalation aerosols according to the invention are also known from the prior art. Suitable propellant gases are selected from among hydrocarbons such as n-propane, n-butane or isobutane and halohydrocarbons such as preferably chlorinated and fluorinated derivatives of methane, ethane, propane, butane, cyclopropane or cyclobutane. The propellant gases mentioned above may be used on their own or in mixtures thereof. Particularly preferred propellant gases are halogenated alkane derivatives selected from TG11, TG12, TG 134a (1,1,1,2-tetrafluoroethane), TG227 (1,1,1,2,3,3,3-heptafluoropropane) and mixtures thereof, the propellant gases TG134a, TG227 and mixtures thereof being preferred.

The inhalation aerosols containing propellant gas according to the invention may contain up to 5% (w/w) of active substance. Aerosols according to the invention contain, for example, 0.002 to 5 wt.-%, 0.01 to 3 wt.-%, 0.015 to 2 wt.-%, 0.1 to 2 wt.-%, 0.5 to 2 wt.-% or 0.5 to 1 wt.-% of the pharmaceutical active substance. Inhalable aerosols with an active substance concentration in this range may be prepared by controlled reconstitution of the powders according to the invention in a corresponding amount of solvent.

The propellant-driven inhalation aerosols according to the invention mentioned above may be administered using inhalers known in the art (MDIs=metered dose inhalers). Reference may be made here to the Ventolin® (Ventolin Pharmacy) or the inhalers described in U.S. Pat. No. 5,32,094 or U.S. Pat. No. 5,672,581. Accordingly, in another aspect, the present invention relates to pharmaceutical compositions in the form of propellant-driven aerosols as hereinbefore described combined with one or more inhalers suitable for administering these aerosols. In addition, the present invention relates to inhalers which are characterised in that they contain the propellant gas-containing aerosols described above according to the invention.

The present invention also relates to cartridges which are fitted with a suitable valve and can be used in a suitable inhaler and which contain one of the above-mentioned propellant gas-containing inhalation aerosols according to the invention. Suitable cartridges and methods of filling these cartridges with the inhalable aerosols containing propellant gas according to the invention are known from the prior art.

The powders according to the invention may also be reconstituted in propellant-free inhalable solutions or suspensions. Corresponding propellant-free inhalable solutions contain for example aqueous or alcoholic, preferably ethanolic solvents, optionally ethanolic solvents mixed with aqueous solvents. In the case of aqueous/ethanolic solvent mixtures the relative proportion of ethanol compared with water is not limited but the maximum is preferably up to 70 percent by volume, more particularly up to 60 percent by volume of ethanol. The remainder of the volume is made up of water. Co-solvents and/or other excipients as described above may be added to the propellant-free inhalable solutions according to the invention. Preferred co-solvents are those which contain hydroxyl groups or other polar groups, e.g. alcohols—particularly isopropyl alcohol, glycols—particularly propyleneglycol, polyethyleneglycol, polypropyleneglycol, glycolether, glycerol, polyoxyethylene alcohols and polyoxyethylene fatty acid esters. The terms excipients and additives in this context denote any pharmacologically acceptable substance which is not an active substance but which can be formulated with the active substance or substances in the pharmacologically suitable solvent in order to improve the qualitative properties of the active substance formulation. Preferably, these substances have no pharmacological effect or, in connection with the desired therapy, no appreciable or at least no undesirable pharmacological effect. The excipients and additives include, in addition to those described above, for example, surfactants such as soya lecithin, oleic acid, sorbitan esters, such as polysorbates, polyvinylpyrrolidone, other stabilisers, complexing agents, antioxidants and/or preservatives which guarantee or prolong the shelf life of the finished pharmaceutical formulation, flavourings, vitamins and/or other additives known in the art. The additives also include pharmacologically acceptable salts such as sodium chloride as isotonic agents. The preferred excipients include antioxidants such as ascorbic acid, for example, provided that it has not already been used to adjust the pH, vitamin A, vitamin E, tocopherols and similar vitamins and provitamins occurring in the human body. Preservatives may be used to protect the formulation from contamination with pathogens. Suitable preservatives are those which are known in the art, particularly cetyl pyridinium chloride, benzalkonium chloride or benzoic acid or benzoates such as sodium benzoate in the concentration known from the prior art. The preservatives mentioned above are preferably present in concentrations of up to 50 mg/100 ml, more preferably between 5 and 20 mg/100 ml. Accordingly, the present invention also includes propellant-free inhalable aerosols which are prepared by reconstituting the powders according to the invention.

The propellant-free inhalable solutions according to the invention are administered in particular using inhalers of the kind which are capable of nebulising a small amount of a liquid formulation in the therapeutic dose within a few seconds to produce an aerosol suitable for therapeutic inhalation. Within the scope of the present invention, preferred inhalers are those in which a quantity of less than 100 μL, preferably less than 50 μL, more preferably between 10 and 30 μL of active substance solution can be nebulised in preferably one spray action to form an aerosol with an average particle size of less than 20 μm, preferably less than 10 μm, such that the inhalable part of the aerosol corresponds to the therapeutically effective quantity.

An apparatus of this kind for propellant-free delivery of a metered quantity of a liquid pharmaceutical composition for inhalation is described for example in International Patent Application WO 91/14468 and also in WO 97/12687 (cf. in particular FIGS. 6a and 6b). Reference is specifically made within the scope of the present invention to the corresponding FIGS. 6a and 6b of WO 97/12687 including the associated parts of the description. The nebulisers (devices) described therein are also known by the name Respimat® (Boehringer Ingelheim Pharma). Because of its cylindrical shape and handy size of less than 9 to 15 cm long and 2 to 4 cm wide, this device can be carried at all times by the patient. The nebuliser sprays a defined volume of the pharmaceutical formulation using high pressures through small nozzles so as to produce inhalable aerosols.

The preferred atomiser essentially consists of an upper housing part, a pump housing, a nozzle, a locking mechanism, a spring housing, a spring and a storage container, characterised by
a pump housing which is secured in the upper housing part and which comprises at one end a nozzle body with the nozzle or nozzle arrangement,
a hollow plunger with valve body,
a power takeoff flange in which the hollow plunger is secured and which is located in the upper housing part,
a locking mechanism situated in the upper housing part,
a spring housing with the spring contained therein, which is rotatably mounted on the upper housing part by means of a rotary bearing,
a lower housing part which is fitted onto the spring housing in the axial direction.

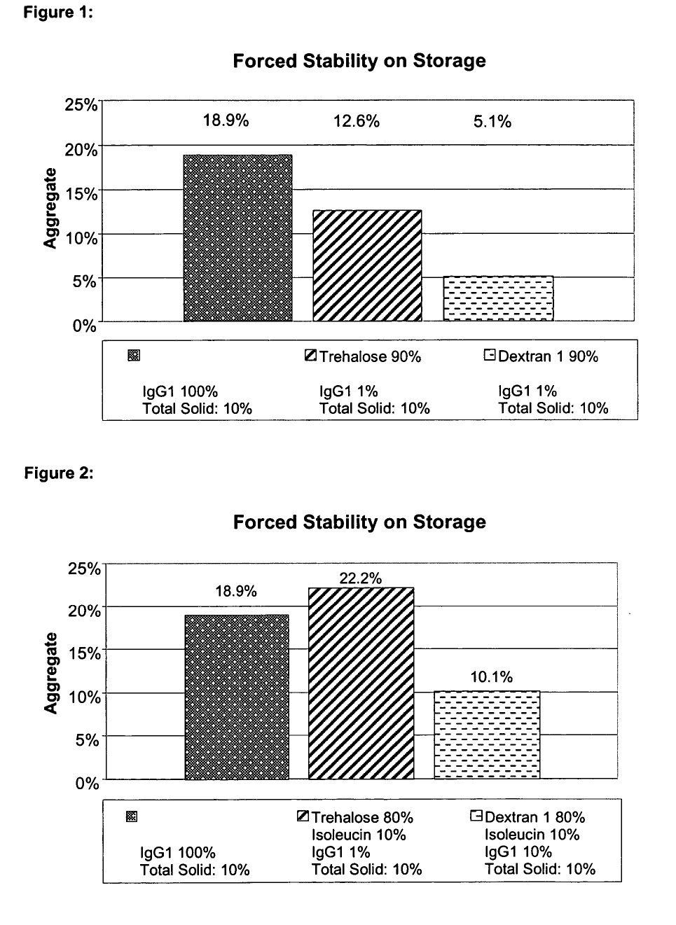

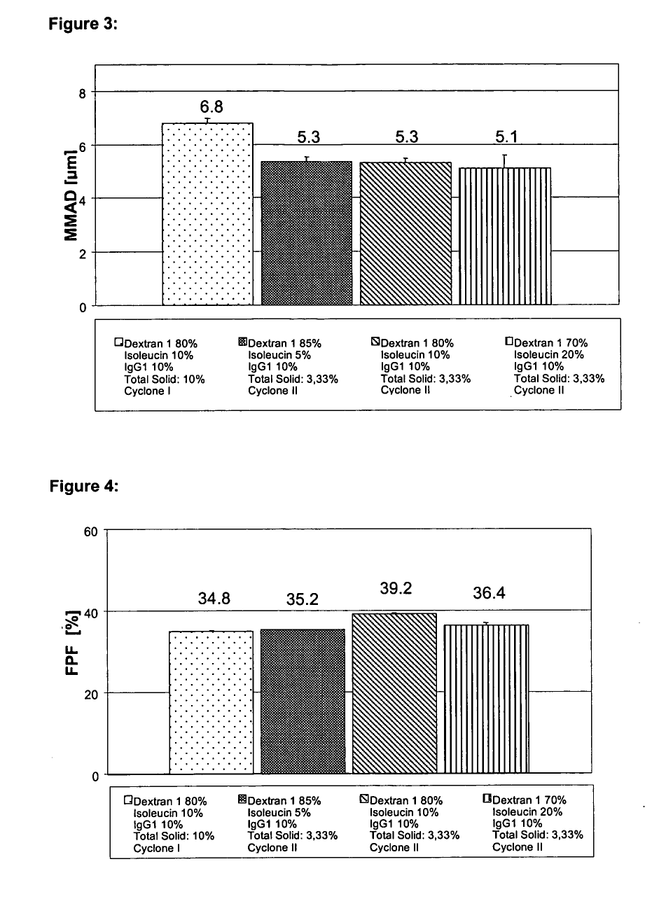

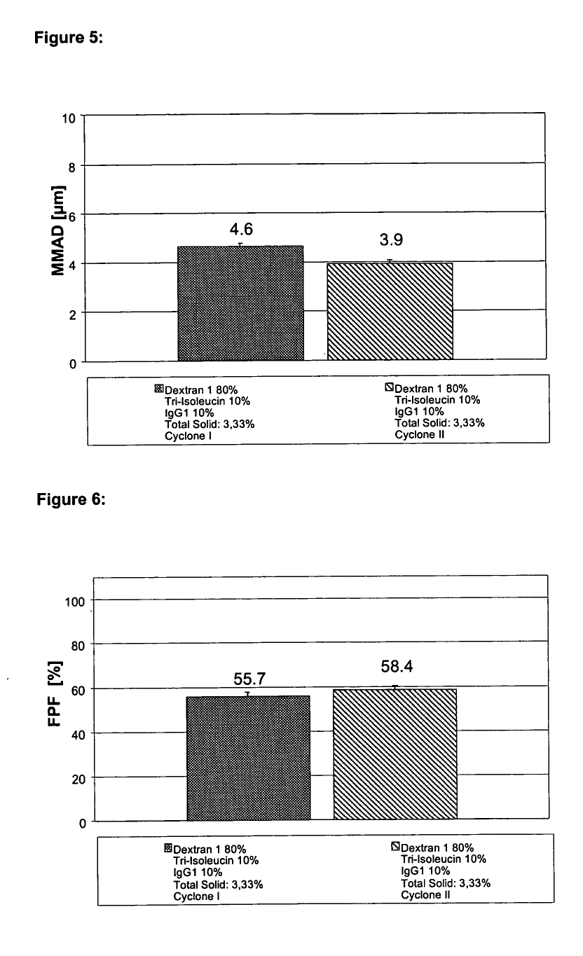

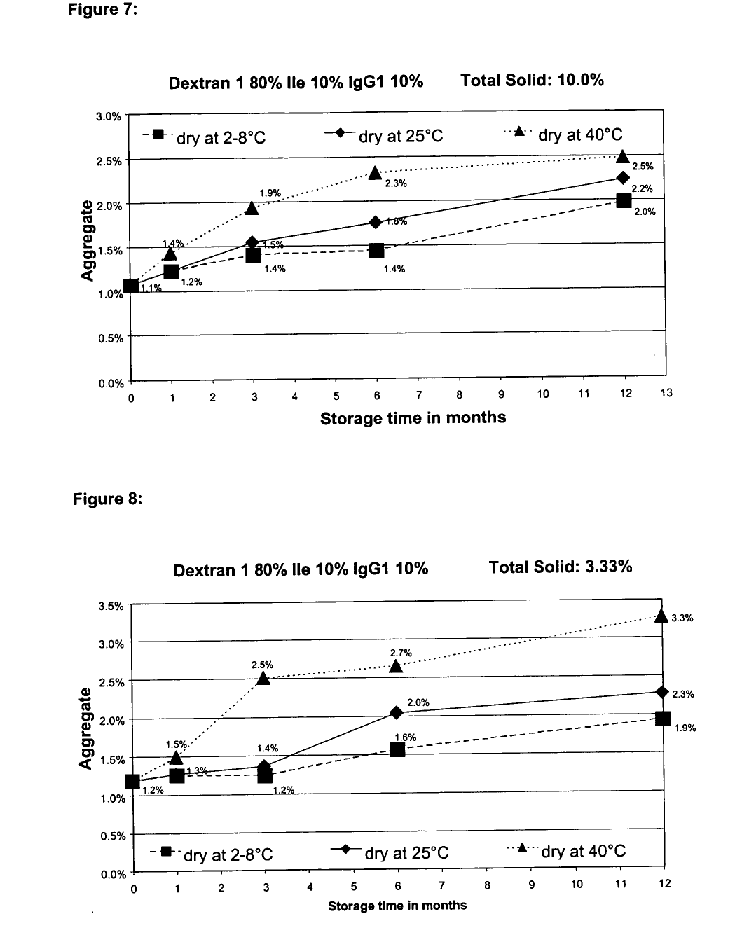

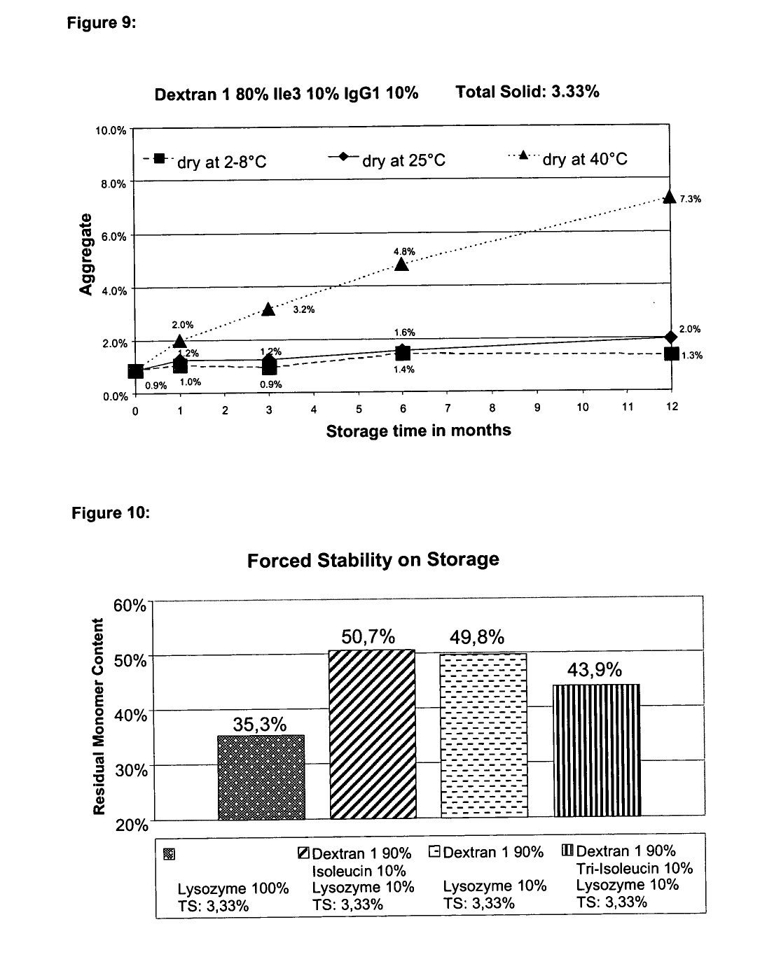

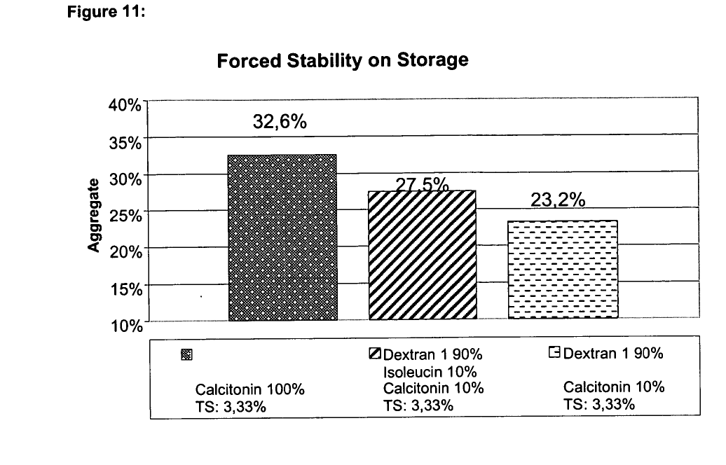

The hollow plunger with valve body corresponds to a device disclosed in WO 97/12687. It projects partially into the cylinder of the pump housing and is axially movable within the cylinder. Reference is made in particular to FIGS. 1 to 4, especially FIG. 3, and the relevant parts of the description. The hollow plunger with valve body exerts a pressure of 5 to 60 MPa (about 50 to 600 bar), preferably 10 to 60 MPa (about 100 to 600 bar) on the fluid, the measured amount of active substance solution, at its high pressure end at the moment when the spring is actuated. Volumes of 10 to 50 microlitres are preferred, while volumes of 10 to 20 microlitres are particularly preferred and a volume of 15 microlitres per spray is most particularly preferred.

The valve body is preferably mounted at the end of the hollow plunger facing the valve body.

The nozzle in the nozzle body is preferably microstructured, i.e. produced by microtechnology. Microstructured nozzle bodies are disclosed for example in WO-94/07607; reference is hereby made to the contents of this specification, particularly FIG. 1 disclosed therein and the associated description. The nozzle body consists for example of two sheets of glass and/or silicon firmly joined together, at least one of which has one or more microstructured channels which connect the nozzle inlet end to the nozzle outlet end. At the nozzle outlet end there is at least one round or non-round opening 2 to 10 microns deep and 5 to 15 microns wide, the depth preferably being 4.5 to 6.5 microns while the length is preferably 7 to 9 microns. In the case of a plurality of nozzle openings, preferably two, the directions of spraying of the nozzles in the nozzle body may extend parallel to one another or may be inclined relative to one another in the direction of the nozzle opening. In a nozzle body with at least two nozzle openings at the outlet end the directions of spraying may be inclined at an angle of 20 to 160° to one another, preferably 60 to 150°, most preferably 80 to 100°. The nozzle openings are preferably arranged at a spacing of 10 to 200 microns, more preferably at a spacing of 10 to 100 microns, most preferably 30 to 70 microns. Spacings of 50 microns are most preferred.

The directions of spraying will therefore meet in the vicinity of the nozzle openings.

The liquid pharmaceutical preparation strikes the nozzle body with an entry pressure of up to 600 bar, preferably 200 to 300 bar, and is atomised into an inhalable aerosol through the nozzle openings. The preferred particle or droplet sizes of the aerosol are up to 20 microns, preferably 3 to 10 microns.

The locking mechanism contains a spring, preferably a cylindrical helical compression spring, as a store for the mechanical energy. The spring acts on the power takeoff flange as an actuating member the movement of which is determined by the position of a locking member. The travel of the power takeoff flange is precisely limited by an upper and lower stop. The spring is preferably biased, via a power step-up gear, e.g. a helical thrust gear, by an external torque which is produced when the upper housing part is rotated counter to the spring housing in the lower housing part. In this case, the upper housing part and the power takeoff flange have a single or multiple V-shaped gear.

The locking member with engaging locking surfaces is arranged in a ring around the power takeoff flange. It consists, for example, of a ring of plastic or metal which is inherently radially elastically deformable. The ring is arranged in a plane at right angles to the atomiser axis. After the biasing of the spring, the locking surfaces of the locking member move into the path of the power takeoff flange and prevent the spring from relaxing. The locking member is actuated by means of a button. The actuating button is connected or coupled to the locking member. In order to actuate the locking mechanism, the actuating button is moved parallel to the annular plane, preferably into the atomiser; this causes the deformable ring to deform in the annular plane. Details of the construction of the locking mechanism are given in WO 97/20590.

The lower housing part is pushed axially over the spring housing and covers the mounting, the drive of the spindle and the storage container for the fluid.

When the atomiser is actuated the upper housing part is rotated relative to the lower housing part, the lower housing part taking the spring housing with it. The spring is thereby compressed and biased by means of the helical thrust gear and the locking mechanism engages automatically. The angle of rotation is preferably a whole-number fraction of 360 degrees, e.g. 180 degrees. At the same time as the spring is biased, the power takeoff part in the upper housing part is moved along by a given distance, the hollow plunger is withdrawn inside the cylinder in the pump housing, as a result of which some of the fluid is sucked out of the storage container and into the high pressure chamber in front of the nozzle.

If desired, a number of exchangeable storage containers which contain the fluid to be atomised may be pushed into the atomiser one after another and used in succession. The storage container contains the aqueous aerosol preparation according to the invention.

The atomising process is initiated by gently pressing the actuating button. As a result, the locking mechanism opens up the path for the power takeoff member. The biased spring pushes the plunger into the cylinder of the pump housing. The fluid leaves the nozzle of the atomiser in atomised form.

Further details of construction are disclosed in PCT Applications WO 97/12683 and WO 97/20590, to the contents of which reference is hereby made.

The components of the atomiser (nebuliser) are made of a material which is suitable for its purpose. The housing of the atomiser and, if its operation permits, other parts as well are preferably made of plastics, e.g. by injection moulding. For medicinal purposes, physiologically safe materials are used.

FIGS. 6a/b of WO 97/12687, including the associated description to which reference is hereby made once more, show a corresponding nebuliser (Respimat®). This is particularly suitable for administering the propellant-free inhalable aerosols according to the invention.

FIG. 6 a of WO 97/12687 shows a longitudinal section through the atomiser with the spring under tension, FIG. 6b of WO 97/12687 shows a longitudinal section through the atomiser with the spring released. The upper housing part (51) contains the pump housing (52), on the end of which is mounted the holder (53) for the atomiser nozzle. In the holder is the nozzle body (54) and a filter (55). The hollow piston (57) fixed in the power take-off flange (56) of the locking clamping mechanism projects partly into the cylinder of the pump housing. At its end the hollow piston carries the valve body (58).

The hollow piston is sealed off by the gasket (59). Inside the upper housing part is the stop (60) on which the power take-off flange rests when the spring is relaxed. Located on the power take-off flange is the stop (61) on which the power take-off flange rests when the spring is under tension. After the tensioning of the spring, the locking member (62) slides between the stop (61) and a support (63) in the upper housing part. The actuating button (64) is connected to the locking member. The upper housing part ends in the mouthpiece (65) and is closed off by the removable protective cap (66). The spring housing (67) with compression spring (68) is rotatably mounted on the upper housing part by means of the snap-fit lugs (69) and rotary bearings. The lower housing part (70) is pushed over the spring housing. Inside the spring housing is the replaceable storage container (71) for the fluid (72) which is to be atomised. The storage container is closed off by the stopper (73), through which the hollow piston projects into the storage container and dips its end into the fluid (supply of active substance solution). The spindle (74) for the mechanical counter is mounted on the outside of the spring housing. The drive pinion (75) is located at the end of the spindle facing the upper housing part. On the spindle is the slider (76).

If the formulation according to the invention is nebulised using the method described above (Respimat®), the mass expelled, in at least 97%, preferably at least 98% of all the actuations of the inhaler (puffs), should correspond to a defined quantity with a range of tolerance of not more than 25%, preferably 20% of this quantity. Preferably, between 5 and 30 mg, more preferably between 5 and 20 mg of formulation are delivered as a defined mass per puff.

However, the formulation according to the invention can also be nebulised using inhalers other than those described above, for example jet-stream inhalers or other stationary nebulisers.

Accordingly, in another aspect, the present invention relates to pharmaceutical compositions in the form of propellant-free inhalable solutions or suspensions as hereinbefore described in conjunction with a device suitable for administering these formulations, preferably in conjunction with the Respimat®. Preferably the present invention is directed to propellant-free Inhalable solutions or suspensions, containing one of the powders according to the invention, in conjunction with the device known as a Respimat®. Moreover the present invention relates to the above-mentioned devices for inhalation, preferably the Respimat®, characterised in that they contain the propellant-free inhalable solutions or suspensions according to the invention as described above.

According to the invention, inhalable solutions containing one of the powders according to the invention as described herein in a single preparation are preferred.

The propellant-free inhalable solutions or suspensions according to the invention may take the form of concentrates or sterile inhalable solutions or suspensions ready for use, as well as the above-mentioned solutions and suspensions designed for use in the Respimat®. Formulations ready for use may be produced from the concentrates, for example, by the addition of isotonic saline solutions. Sterile formulations ready for use may be administered using energy-operated fixed or portable nebulisers which produce inhalable aerosols by means of ultrasound or compressed air by the Venturi principle or other principles.

Accordingly, in another aspect, the present invention relates to pharmaceutical compositions in the form of propellant-free inhalable solutions or suspensions as described hereinbefore which take the form of concentrates or sterile formulations ready for use, combined with a device suitable for administering these solutions, characterised in that the device is an energy-operated free-standing or portable nebuliser which produces inhalable aerosols by means of ultrasound or compressed air by the Venturi principle or other methods.

Other suitable nebulisers for inhaling reconstituted aerosols are the AERx™ (Aradigm), Ultravent® (Mallinkrodt) and AconII® (Maquest Medical Products).

### EXAMPLES

Equipment and Methods

Materials:

A humanised monoclonal antibody with a molecular weight of about 148 kDa was used as IgG1. The antibody is derived from a murine antibody in which the complementarity-determining regions of the murine antibody have been transferred to a human immunoglobulin structure. A chimeric antibody has been produced with 95% human content and 5% murine content. The antibody is expressed by murine myeloma cell lines. The cells are removed by Tangential Flow Microfiltration and the cell-free solution is purified by various chromatographic methods. Other steps include nuclease treatment, treatment at a low pH and nanofiltration. The bulk solution containing the antibodies contains 25 mM histidine and 1.6 mM glycine as buffer and has been concentrated to approx. 100 mg/ml by diafiltration, for the preparation of the solution for spray drying. The bulk for the preparation of the spray solution contained 0.8% aggregates. The finished drug can be stored at 2-8° C. for at least 2 years. Low molecular dextran1 or dextran₁₀₀₀ with a mean molecular weight of about 1000 Da obtained from Amersham Biosciences AB, Uppsalla, Sweden. Trehalose is obtained from Georg Breuer GmbH, Germany. L-isoleucine was obtained from Sigma-Aldrich Chemie GmbH, Germany. Triisoleucine was obtained from Iris Biotech GmbH, Germany. Chicken albumin lysozyme (lysozyme), 135500 U/mg, was obtained from SERVA Electrophoresis GmbH, Germany. Synthetic salmon calcitonin (calcitonin) was obtained from Biotrend Chemikalien GmbH, Germany.

Spray-DRYING with Büchi B-290

The spray-drying was done using a Büchi Mini Spray Dryer B-290 made by Messrs Büchi Labortechnik (AG, CH). The spray-drying of the formulations was carried out chiefly as described in the “Spray Drying Handbook”, 5th Edition., K. Masters, John Wiley and Sons, Inc., NY, N.Y. (1991):

The spray drier is made up of a heating system, a filter, an aspirator, a drying tower, a cyclone, temperature sensors for measuring the inlet and outlet temperature and a collecting vessel. The solution to be sprayed is pumped into the two-substance nozzle by means of a peristaltic pump. There, the solution is atomised into small drops using compressed air. The drying in the spray tower is done using heated air which is aspirated through the spray tower by the direct current method by means of the aspirator. The product is collected in the collecting vessel after passing through the cyclone.

Two different cyclones were used:

| | |
|---|---|
| Cyclone I: Büchi Cyclone | (product number 4189) |
| Cyclone II: Büchi High-performance Cyclone | (product number 46369) |

The solid content of the spray solutions was 10% (w/v) in 50 ml, 3.33% in 300 ml and 3.33% in 600 ml. The inlet temperature was about 170 to 185° C., the liquid feed rate approx. 3 ml/min, the aspirator flow rate 35 m³/h and the atomiser flow rate 0.67 m³/h. This produced an outlet temperature of about 80-95° C.

X-Ray Diffractometry (Wide-Angle X-Ray Diffractometry (WAXS)):

In order to determine the crystallinity of the dried samples the samples were investigated with a Seifert X-ray diffractometer XRD 3000 TT (Messrs Seifert, Ahrensburg, Del.) in a chamber at a controlled temperature of 22° C. The X-ray tube Cu anode, Cu-Kα radiation with λ=0.15418 mm (Ni primary filter), was operated at an anode voltage of 40 kV and a current strength of 30 mA. After the sample dish had been placed in the apparatus the sample was measured in the range from 5 to 40° at a scan rate of 2θ=0.05° with 2 sec measuring time at each angle.

The powder diffractograms were taken with the ScanX—Rayflex application, Version 3.07 device XRD 3000 (Scan), or the Rayflex Version 2.1, 1996 (Analysis) on the SC 1000 V detector.

Size Exclusion Chromatography (SEC-HPLC):

a) Soluble IgG1 Protein Aggregates

SEC-HPLC was used to quantify IgG1-protein aggregates in the reconstituted powders. The SEC-HPLC was carried out with a HP1090 made by Messrs Agilent. The column used for separation was a TSK3000SWXL column (300×7.8 mm) made by Messrs Tosoh Biosep (Tosoh Bioscience, Stuttgart, Del.). The eluant used was a buffer consisting of 0.1M di-sodium hydrogen phosphate-dihydrate, 0.1M sodium sulphate which was dewatered and adjusted to pH 6.8 with ortho-phosphoric acid 85%. The amount of sample put in was 25 μl at a protein concentration of 2-10 mg/ml. The protein was detected using a diode array detector made by Messrs Agilent at 280 nm. The chromatographs were evaluated using the Chemstation software made by Agilent.

b) Soluble Calcitonin Protein Aggregates

In order to quantify calcitonin-protein aggregates in the reconstituted powders SEC-HPLC was carried out. The SEC-HPLC was carried out using an HP1100 made by Messrs Agilent. The column used for separation was a TSK3000SWXL column (300×7.8 mm) made by Messrs Tosoh Biosep (Tosoh Bioscience, Stuttgart, Del.). The eluant used was a buffer consisting of 0.25 sodium sulphate with a pH of about 6 (Windisch et al. 1997). The amount of sample put in was 20 μl at a protein concentration of 0.5-2 mg/ml. The protein was detected using a UV detector made by Messrs Agilent at 210 nm. The chromatographs were evaluated using the HP-Chemstation software made by Messrs Agilent.

c) Lysozyme Residual Monomer Content

In order to quantify the lysozyme residual monomer content in the reconstituted lysozyme formulations a modified SEC-HPLC was carried out (van de Weert, 2000). The SEC-HPLC was carried out using an HP1100 made by Messrs Agilent. The column used for separation was a TSK2000SWXL column (300×7.8 mm) made by Messrs Tosoh Biosep (Tosoh Bioscience, Stuttgart, Del.). The eluant used was a buffer consisting of 0.05 M disodium hydrogen phosphate-dihydrate and 0.2 M sodium chloride, adjusted to pH 7.0 with ortho-phosphoric acid 85%. The amount of sample put in was 25 μl at a protein concentration of 2-10 mg/ml. The protein was detected using a UV detector made by Messrs Agilent at 280 nm. The chromatographs were evaluated using the Agilent Chemstation software made by Messrs Agilent.

In order to evaluate the formulations, the soluble monomer remaining was quantified by the following method. First, a calibrating line was drawn using lysozyme standard solutions with concentrations of 2.5 mg/ml, 5.0 mg/ml and 10 mg/ml. The AUC of the monomer peaks was studied in relation to the corresponding lysozyme concentrations in the standard solution under investigation.

The residual monomer content of the various lysozyme formulations under investigation was calculated using the calibrating line. The higher the residual monomer content of a formulation, the better the protein stability.

Determining the Particle Size (MMD):

The Mass Median Diameter or the mean particle size of the particles was determined using the Sympatech Helos made by Messrs Sympatech GmbH (Clausthal-Zellerfeld, Del.). The measuring principle is based on laser diffraction, using a helium neon laser. 1-3 mg of powder are dispersed with an air pressure of 2 bar, and passed through a parallel laser beam in front of the Fourier lens (50 mm). The particle size distribution is evaluated using a Fraunhofer model. Two measurements were carried out on each powder.

Mass Median Diameter (MMAD) and Fine Particle Fraction (FPF)

For the measurements, 12-18 mg of powder were transferred into hard gelatine capsules (size 3) and placed in the HandiHaler (powder inhaler made by Messrs Boehringer Ingelheim). Using an adapter the HandiHaler was coupled to the USP EP/throat of the impactor inlet of the measuring device and the powder was delivered at a rate of 39.0 l/min with an intake time of 6.15 sec. The air throughput was controlled by means of an external controlling wall. At least three capsules were measured for each powder.

The APS 3321 of Messrs TSI Inc., MN, USA is used in conjunction with the Impactorinlet 3306 to simultaneously measure the aerodynamic particle size (MMAD) by measuring the time of flight and the fine particle fraction (FPF) using a one-step impactor (effective cut off diameter at 39L/min: 5.0 μm). After being expelled through the EP/USP Throat or Sample Induction Port the powder reaches a thin capillary where 0.2% of the powder can be removed under isokinetic conditions in order to measure the time of flight. The time of flight is measured after passing the capillary through 2 laser beams which detect the time of flight for a defined distance analogously to a light barrier. As a result, a numerical distribution is obtained which is then converted into a mass distribution and thus into the Mass Median Aerodynamic Diameter (MMAD).

The remaining 99.8% of the powder population which has travelled past the capillary is then separated off using the one-step impactor. The fraction larger than 5.0 μm is deposited on a baffle plate in the impactor as a result of mass inertia. The fine particle fraction (FPF) follows the air current and is finally deposited on a deep filter. The fine particle fraction is determined by gravimetry. The fine particle fraction is calculated from the amount of powder deposited on the filter relative to the total amount of powder used, i.e. the powder weighed out for each capsule.

Residual Water Content:

The residual water content in the dried products was determined by coulometric titration (Metrohm 737 KF Coulometer with 703 titration stand, Germany). For the measurement, powder was dissolved or dispersed in methanol (Hydranal—Methanol dry, VWR/Merck Eurolab). The measuring solution (Hydranal—Coulomat solution, VWR/Merck Eurolab) of the Metrohm Coulometer was adjusted at the start of the measurements, i.e. the measuring solution was calibrated to a zero content of water. The sample was injected into the titration cell and measured.

Determining Stability:

The powders were investigated for different stabilities after spray-drying. In the case of IgG1 and calcitonin the percentage amount of protein aggregates was used as the measurement of stability of the formulations. In the case of lysozyme the percentage amount of the residual monomer content was used as the measurement of stability of the formulations. The innovative excipients described in the invention were compared with the pure protein formulation and optionally an analogous trehalose formulation as reference. Analysis to detect any aggregates was carried out with a validated size exclusion chromatography (SEC-HPLC) with UV detection (DAD). For this the pretreated powders were first reconstituted in highly purified water (pH 6 to 8). Selected formulations were investigated for their stability after one weeks open storage at about 40° C. and about 75% relative humidity (40° C., 75% rh) in open glass vials (forced storage stability).

Selected formulations were stored after spray-drying and vacuum drying under nitrogen in sealed glass vials at 2-8° C., 25° C. and 40° C. The formulations were removed after one, three, six and twelve months and tested for their stability (stability over 1 year).

### Example 1

### Spray-Drying a 10% (w/v) IgG1 Formulation

Pure IgG1 in a concentration of about 109 mg/ml, formulated in a glycine histidine buffer, pH 6 (see Materials), was diluted with demineralised water (pH about 7.5) to a content of 100 mg/ml and spray-dried in the absence of any other excipients as described above using the Cyclone I. The volume of the solution was 50 ml. The content of aggregates was investigated as described above. After forced storage the solution of the reconstituted powder contained about 18.9% aggregates.

### Spray-Drying a Formulation Containing 9% (w/v) Trehalose 1% (w/v) IgG1

4.5 g trehalose was dissolved in about 40 ml of demineralised water (pH about 7.5). Next, about 4.6 ml of pure IgG1 with a concentration of about 109 mg/ml, formulated in a glycine histidine buffer pH 6 (see Materials), was added and diluted to a volume of 50 ml with demineralised water (pH about 7.5). The solution thus obtained contains about 9% (w/v) excipient or matrix and 1% (w/v) protein and was spray-dried as described above using the Cyclone I. The content of aggregates was investigated as described above. After forced storage the solution of the reconstituted powder contained about 12.6% aggregates.

### Spray-drying a formulation containing 9% (w/v) dextran₁₀₀₀ 1% (w/v) IgG1

4.5 g dextran₁₀₀₀ was dissolved in about 40 ml of demineralised water (pH about 7.5). Next, about 4.6 ml of pure IgG1 with a concentration of about 109 mg/ml, formulated in a glycine histidine buffer pH 6 (see Materials), was added and diluted to a volume of 50 ml with demineralised water (pH about 7.5). The solution thus obtained contains about 9% (w/v) excipient or matrix and 1% (w/v) protein and was spray-dried as described above using the Cyclone I. The content of aggregates was investigated as described above. The following aggregate contents were obtained for the storage stability. After forced storage the solution of the reconstituted powder contained about 5.1% aggregates.

### Spray-Drying a Formulation Containing 2.00% (w/v) dextran₁₀₀₀ 1.33% (w/v) IgG1

3.0 g dextran₁₀₀₀ was dissolved in about 120 ml of demineralised water (pH about 7.5). Next, about 19.5 ml of pure IgG1 with a concentration of about 102.8 mg/ml, formulated in a glycine histidine buffer pH 6 (see Materials), was added and diluted to a volume of 150 ml with demineralised water (pH about 7.5). The solution thus obtained contains about 2.0% (w/v) excipient or matrix and 1.33% (w/v) protein and was spray-dried as described above using the Cyclone II. The content of aggregates was investigated as described above. The following aggregate contents were obtained for the storage stability. After forced storage the solution of the reconstituted powder contained about 11.1% aggregates.

### Example 2

### Spray-Drying a Formulation Containing 8% (w/v) Trehalose 1% (w/v) L-isoleucine 1% (w/v) IgG1

4 g trehalose and 0.5 g L-isoleucine were dissolved in an ultrasound bath in about 40 ml of demineralised water (pH about 7.5). Next, about 4.6 ml of pure IgG1 with a concentration of about 109 mg/ml, formulated in a glycine histidine buffer pH 6 (see Materials), was added and diluted to a volume of 50 ml with demineralised water (pH about 7.5). The solution thus obtained contains about 9% (w/v) excipient or matrix and 1% (w/v) protein and was spray-dried as described above using the Cyclone I. The content of aggregates was investigated as described above. After forced storage the solution of the reconstituted powder contained about 22.2% aggregates.

### Spray-Drying a Formulation Containing 8% (w/v) dextran₁₀₀₀ 1% (w/v) L-isoleucine 1% (w/v) IgG1

4 g dextran₁₀₀₀ and 0.5 g L-isoleucine were dissolved in an ultrasound bath in about 40 ml of demineralised water (pH about 7.5). Next, about 4.6 ml of pure IgG1 with a concentration of about 109 mg/ml, formulated in a glycine histidine buffer pH 6 (see Materials), was added and diluted to a volume of 50 ml with demineralised water (pH about 7.5). The solution thus obtained contains about 9% (w/v) excipient or matrix and 1% (w/v) protein and was spray-dried as described above using the Cyclone I. The content of aggregates was investigated as described above. After forced storage the solution of the reconstituted powder contained only about 10.1% aggregates. In a second larger mixture an analogous formulation (20g solid and 200 ml volume) was spray-dried under the same conditions. The content of aggregates was investigated as described above. After 12 months storage at 40° C. (1 years stability) the solution of the reconstituted powder contained about 2.5% aggregates. After 3 months storage at 25° C. (3 months stability) the solution of the reconstituted powder contained about 2.2% aggregates. After 3 months storage at 2-8° C. (3 months stability) the solution of the reconstituted powder contained about 2.0% aggregates. The powder obtained was measured for MMD, MMAD and FPF. The MMD of the powder was determined as described above. The MMD of the powder was 5.11 μm. The MMAD and FPF of the powder were determined as described above. The MMAD was 6.8 μm and the Fine Particle Fraction was 34.8% relative to the weight of powder placed in the capsule.

### Spray-Drying a Formulation Containing 2.833% (w/v) dextran₁₀₀₀, 0.166% (w/v) L-isoleucine, 0.33% (w/v) IgG1

8.5 g dextran₁₀₀₀ and 0.5 g L-isoleucine were dissolved in an ultrasound bath in about 280 ml of demineralised water (pH about 7.5). Next, about 9.7 ml of pure IgG1 with a concentration of about 102.8 mg/ml, formulated in a glycine histidine buffer pH 6 (see Materials), was added and diluted to a volume of 300 ml with demineralised water (pH about 7.5). The solution thus obtained contains about 3% (w/v) excipient or matrix and 0.33% (w/v) protein and was spray-dried as described above using the Cyclone II.

The content of aggregates was investigated as described above. After forced storage the solution of the reconstituted powder contained about 6.3% aggregates. The MMD of the powder was determined as described above. The MMD of the powder was 2.98 μm. The MMAD and FPF of the powder was determined as described above. The MMAD was 5.3 μm and the Fine Particle Fraction was 35.2% relative to the weight of powder placed in the capsule.

### Spray-Drying a Formulation Containing 2.66% (w/v) dextran₁₀₀₀, 0.33% (w/v) L-isoleucine, 0.33% (w/v) IgG1

8.0 g dextran₁₀₀₀ and 1 g L-isoleucine were dissolved in an ultrasound bath in about 280 ml of demineralised water (pH about 7.5). Next, about 9.7 ml of pure IgG1 with a concentration of about 102.8 mg/ml, formulated in a glycine histidine buffer pH 6 (see Materials), was added and diluted to a volume of 300 ml with demineralised water (pH about 7.5). The solution thus obtained contains about 3% (w/v) excipient or matrix and 0.33% (w/v) protein and was spray-dried as described above using the Cyclone II.

The content of aggregates was investigated as described above. After forced storage the solution of the reconstituted powder contained about 7.1% aggregates. After 12 months storage at 40° C. (1 years stability) the solution of the reconstituted powder contained about 3.3% aggregates. After 12 months storage at 25° C. (1 years stability) the solution of the reconstituted powder contained about 2.3% aggregates. After 12 months storage at 2-8° C. (1 years stability) the solution of the reconstituted powder contained about 1.9% aggregates. The MMD of the powder was determined as described above. The MMD of the powder was 2.75 μm. The MMAD and FPF of the powder was determined as described above. The MMAD was 5.3 μm and the Fine Particle Fraction was 39.2% relative to the weight of powder placed in the capsule.

### Spray-Drying a Formulation Containing 2.33% (w/v) dextran₁₀₀₀, 0.66% (w/v) L-isoleucine, 0.33% (w/v) IgG1

7.0 g dextran₁₀₀₀ and 2 g L-isoleucine were dissolved in an ultrasound bath in about 280 ml of demineralised water (pH about 7.5). Next, about 9.7 ml of pure IgG1 with a concentration of about 102.8 mg/ml, formulated in a glycine histidine buffer pH 6 (see Materials), was added and diluted to a volume of 300 ml with demineralised water (pH about 7.5). The solution thus obtained contains about 3% (w/v) excipient or matrix and 0.33% (w/v) protein and was spray-dried as described above using the Cyclone II.

The content of aggregates was investigated as described above. After forced storage the solution of the reconstituted powder contained about 10.6% aggregates. The MMD of the powder was determined as described above. The MMD of the powder was 2.71 μm. The MMAD and FPF of the powder was determined as described above. The MMAD was 5.1 μm and the Fine Particle Fraction was 36.4% relative to the weight of powder placed in the capsule.

### Example 3

### Spray-Drying a Formulation Containing 2.66% (w/v) dextran₁₀₀₀, 0.33% (w/v) Triisoleucine and 0.33% (w/v) IgG1

16.0 g dextran₁₀₀₀ and 2 g triisoleucine were dissolved in an ultrasound bath in about 560 ml of demineralised water (pH about 7.5). Next, about 20.7 ml of pure IgG1 with a concentration of about 96.55 mg/ml, formulated in a glycine histidine buffer pH 6 (see Materials), was added and diluted to a volume of 600 ml with demineralised water (pH about 7.5). The solution thus obtained contains about 3% (w/v) excipient or matrix and 0.33% (w/v) protein and was spray-dried as described above using the Cyclone I. The content of aggregates was investigated as described above. After 3 months storage at 40° C. (3 months stability) the solution of the reconstituted powder contained about 3.2% aggregates. After 3 months storage at 25° C. (3 months stability) the solution of the reconstituted powder contained about 1.2% aggregates. After 3 months storage at 2-8° C. (3 months stability) the solution of the reconstituted powder contained about 0.9% aggregates. After 12 months storage at 40° C. (1 years stability) the solution of the reconstituted powder contained about 7.3% aggregates. After 12 months storage at 25° C. (1 years stability) the solution of the reconstituted powder contained about 2.0% aggregates. After 12 months storage at 2-8° C. (1 years stability) the solution of the reconstituted powder contained about 1.3% aggregates. The MMAD and FPF of the powder was determined as described above. The MMAD was 4.6 μm and the Fine Particle Fraction was 55.7% relative to the weight of powder placed in the capsule.

### Spray-Drying a Formulation Containing 2.66% (w/v) dextran₁₀₀₀, 0.33% (w/v) Triisoleucine and 0.33% (w/v) IgG1

8.0 g dextran₁₀₀₀ and 1 g triisoleucine were dissolved in an ultrasound bath in about 280 ml of demineralised water (pH about 7.5). Next, about 10.36 ml of pure IgG1 with a concentration of about 96.55 mg/ml, formulated in a glycine histidine buffer pH 6 (see Materials), was added and diluted to a volume of 300 ml with demineralised water (pH about 7.5). The solution thus obtained contains about 3% (w/v) excipient or matrix and 0.33% (w/v) protein and was spray-dried as described above using the Cyclone II. The stability was not measured as the formulation is exactly the same as that described immediately above. The use of a different cyclone has no effect on the protein stability. The MMD of the powder was determined as described above. The MMD of the powder was 2.96 μm. The MMAD and FPF of the powder was determined as described above. The MMAD was 3.9 μm and the Fine Particle Fraction was 58.4% relative to the weight of powder placed in the capsule.

### Example 4

### Preparation of Other Powders According to the Invention

### Spray-Drying a Formulation Containing 3.33% (w/v) Lysozyme

5 g lysozyme is dissolved in about 140 ml of demineralised water (pH about 7.5) and diluted to a volume of 150 ml with demineralised water (pH about 7.5). The solution thus obtained is spray-dried as described above using the Cyclone II. The residual monomer content was investigated as described above. After forced storage the solution of the reconstituted powder contained a residual monomer content of 35.3%. The MMD of the powder was determined as described above. The MMD of the powder was 3.23 μm. The MMAD and FPF of the powder was determined as described above. The MMAD was 4.0 μm and the Fine Particle Fraction was 70.4% relative to the weight of powder placed in the capsule.

### Spray-Drying a Formulation Containing dextran₁₀₀₀ 3.00% (w/v) Lysozyme 0.33% (w/v) Formulation

9.0 g dextran₁₀₀₀ is dissolved in the ultrasound bath in about 280 ml demineralised water (pH about 7.5). Next, 1 g lysozyme is added, and the mixture is diluted with demineralised water (pH about 7.5) to a volume of 300 ml. The solution thus obtained is spray-dried as described above using the Cyclone II. The residual monomer content was investigated as described above. After forced storage the solution of the reconstituted powder contained a residual monomer content of 49.8%. The MMD of the powder was determined as described above. The MMD of the powder was 2.82 μm. The MMAD and FPF of the powder was determined as described above. The MMAD was 4.2 μm and the Fine Particle Fraction was 34.7% relative to the weight of powder placed in the capsule.

### Spray-Drying a Formulation Containing 2.66% (w/v) dextran₁₀₀₀, 0.33% (w/v) Isoleucine and 0.33% (w/v) Lysozyme

8.0 g dextran₁₀₀₀ and 1 g isoleucine are dissolved in the ultrasound bath in about 280 ml demineralised water (pH about 7.5). Next, 1 g lysozyme is added, and the mixture is diluted with demineralised water (pH about 7.5) to a volume of 300 ml. The solution thus obtained is spray-dried as described above using the Cyclone II.

The residual monomer content was investigated as described above. After forced storage the solution of the reconstituted powder contained a residual monomer content of 50.7%. The MMD of the powder was determined as described above. The MMD of the powder was 3.01 μm. The MMAD and FPF of the powder was determined as described above. The MMAD was 4.2 μm and the Fine Particle Fraction was 36.6% relative to the weight of powder placed in the capsule.

### Spray-Drying a Formulation Containing 2.66% (w/v) dextran₁₀₀₀, 0.33% (w/v) triisoleucine and 0.33% (w/v) lysozyme

8.0 g dextran₁₀₀₀ and 1 g triisoleucine are dissolved in the ultrasound bath in about 280 ml demineralised water (pH about 7.5). Next, 1 g lysozyme is added, and the mixture is diluted with demineralised water (pH about 7.5) to a volume of 300 ml. The solution thus obtained is spray-dried as described above using the Cyclone II.

The residual monomer content was investigated as described above. After forced storage the solution of the reconstituted powder contained a residual monomer content of 43.9%. The MMD of the powder was determined as described above. The MMD of the powder was 2.53 μm. The MMAD and FPF of the powder was determined as described above. The MMAD was 3.2 μm and the Fine Particle Fraction was 58.6% relative to the weight of powder placed in the capsule.

### Spray-drying a Formulation Containing 3.33% (w/v) Calcitonin

1 g calcitonin is dissolved in about 25 ml demineralised water (pH about 7.5) and diluted with demineralised water (pH about 7.5) to a volume of 30 ml. The solution thus obtained is spray-dried as described above using the Cyclone II.

The content of aggregates was investigated as described above. After forced storage the solution of the reconstituted powder contained about 32.6% aggregates. The MMAD and FPF of the powder was determined as described above. The MMAD was 3.9 μm and the Fine Particle Fraction was 59.0% relative to the weight of powder placed in the capsule.

### Spray-Drying a Formulation Containing 3.166% (w/v) dextran₁₀₀₀ and 0.166% (w/v) Calcitonin

4.750 g dextran₁₀₀₀ is dissolved in about 140 ml demineralised water (pH about 7.5) in the ultrasound bath. Then 0.250 g calcitonin is added and diluted with demineralised water (pH about 7.5) to a volume of 150 ml. The solution thus obtained is spray-dried as described above using the Cyclone II.

The content of aggregates was investigated as described above. After forced storage the solution of the reconstituted powder contained about 27.5% aggregates. The MMD of the powder was determined as described above. The MMD of the powder was 3.00 μm. The MMAD and FPF of the powder was determined as described above. The MMAD was 4.6 μm and the Fine Particle Fraction was 42.6% relative to the weight of powder placed in the capsule.

### Spray-Drying a Formulation Containing 2.833% (w/v) dextran₁₀₀₀, 0.33% (w/v) Isoleucine and 0.166% (w/v) Calcitonin

4.250 g dextran₁₀₀₀ and 0.50 g isoleucine are dissolved in about 140 ml demineralised water (pH about 7.5) in the ultrasound bath. Then 0.250 g calcitonin is added and diluted with demineralised water (pH about 7.5) to a volume of 150 ml. The solution thus obtained is spray-dried as described above using the Cyclone II.

The content of aggregates was investigated as described above. After forced storage the solution of the reconstituted powder contained about 23.2% aggregates. The MMD of the powder was determined as described above. The MMD of the powder was 2.86 μm. The MMAD and FPF of the powder was determined as described above. The MMAD was 4.5 μm and the Fine Particle Fraction was 57.1% relative to the weight of powder placed in the capsule.

### Spray-Drying a Formulation Containing 2.866% (w/v) dextran₁₀₀₀, 0.33% (w/v) Triisoleucine and 0.166% (w/v) Calcitonin

4.250 g dextran₁₀₀₀ and 0.50 g triisoleucine are dissolved in about 140 ml demineralised water (pH about 7.5) in the ultrasound bath. Then 0.250 g calcitonin is added and diluted with demineralised water (pH about 7.5) to a volume of 150 ml. The solution thus obtained is spray-dried as described above using the Cyclone II.

The MMD of the powder was determined as described above. The MMD of the powder was 2.60 μm. The MMAD and FPF of the powder was determined as described above.

The MMAD was 3.7 μm and the Fine Particle Fraction was 62.5% relative to the weight of powder placed in the capsule.

1. A Spray-dried powder containing a pharmaceutically active substance and a low-molecular dextran with a molecular weight between 500 and 10,000 Da.

2. The Spray-dried powder according to claim 1 wherein the low-molecular dextran has a molecular weight between 500 and 5,000 Da.

3. The Spray-dried powder according to claim 1 wherein the low-molecular dextran has a molecular weight of 500 to 1,500 Da.

4. The Spray-dried powder according to claim 3 wherein the powder contains between 50 and 99.99% (w/w) relative to its dry mass of low-molecular dextran.

5. The Spray-dried powder according to claim 4 wherein the powder contains between 60 and 99.99% (w/w) relative to its dry mass of low-molecular dextran.

6. The Spray-dried powder according to claim 1 wherein the pharmaceutically active substance is a biological macromolecule.

7. The Spray-dried powder according to claim 6 wherein the biological macromolecule is a polypeptide or protein.

8. The Spray-dried powder according to claim 7 wherein the polypeptide or protein is a growth factor, an enzyme or an antibody.

9. The Spray-dried powder according to claim 1 wherein the amount of pharmaceutically active substance is between 0.01 and 50% (w/w) of the dry mass of the powder.

10. The Spray-dried powder according to claim 3 wherein the dry mass of the spray-dried powder contains at least 50% (w/w) of a low-molecular dextran and up to 50% (w/w) of a biological macromolecule.

11. The Spray-dried powder according to claim 10 wherein the dry mass of the spray-dried powder contains at least 60 (w/w) of a low-molecular dextran and up to 40% (w/w) of a biological macromolecule.

12. The Spray-dried powder according to one of claims 1, 4, 6, 9 or 10 wherein the spray-dried powder contains one or more pharmaceutically acceptable excipients and/or one or more pharmaceutically acceptable salts.

13. The Spray-dried powder according to claim 12 wherein the spray-dried powder contains one or more amino acid(s) as excipient in addition to the low-molecular dextran and the pharmaceuticaly active substance.

14. The Spray-dried powder according to claim 13 wherein the spray-dried powder contains isoleucine as excipient in addition to the low-molecular dextran and the pharmaceutically active substance.

15. The Spray-dried powder according to claim 12 wherein the spray-dried powder contains a tripeptide as excipient in addition to the low-molecular dextran and the pharmaceutical active substance.

16. The Spray-dried powder according to claim 15 wherein the tripeptide is an isoleucine-containing tripeptide.

17. The Spray-dried powder according to claim 15 wherein the tripeptide is triisoleucine.

18. The Spray-dried powder according to claim 12 wherein the dry mass of the spray-dried powder contains at least 50 (w/w) of low-molecular dextran and between 1 and 20% (w/w) of at least one amino acid and/or at least one peptide.

19. The Spray-dried powder according to claim 14 wherein the dry mass of the spray-dried powder contains at least 50% (w/w) of low-molecular dextran and between 10 and 20% (w/w) of isoleucine.

20. The Spray-dried powder according to claim 15 wherein the dry mass of the spray-dried powder contains at least 50% (w/w) of low-molecular dextran and between 1 and 20% (w/w) of a tripeptide.

21. The Spray-dried powder according to one of claim 1 wherein the particles in the powder have a MMAD of between 1 and 10 μm.

22. The Spray-dried powder according to claim 21 wherein the particles in the powder have a MMAD of between 1 and 7.5 μm.

23. The Spray-dried powder according to claim 22 wherein the particles in the powder have a MMAD of between 1 and 5.5 μm.

24. The Spray-dried powder according to one of claims 1 to 3 wherein the spray-dried powder is amorphous.

25. The Spray-dried powder according to one of claims 1 to 3 wherein the spray-dried powder has a glass transition temperature of 45 to 65° C.

26. Spray-dried powder according to claim 25, wherein the spray-dried powder has a glass transition temperature of 55 to 65° C.

27. A Pharmaceutical composition containing a spray-dried powder according to claim 1.

28. A Process for preparing a spray-dried powder according to claim 1 comprising
a) dissolving a pharmaceutical active substance in an aqueous solution/suspension;
b) dissolving a low-molecular dextran in an aqueous solution/suspension;
c) mixing the active substance and low-molecular dextran if the active substance and low-molecular dextran are dissolved in different solutions/suspension; and
d) spraying the solution/suspension containing low-molecular dextran and the pharmaceutical active substance at a temperature below 200/120° C. (inflow/outflow temperature).

29. The Process according to claim 28 wherein the pharmaceutical active substance is a biological macromolecule.

30. The Process according to claim 29 wherein the biological macromolecule is a polypeptide or protein.

31. The Process according to claim 30 wherein the polypeptide or protein is a growth factor, an enzyme or an antibody.

32. The Process according to one of claims 28 wherein the solution or suspension additionally contains one or more excipients and/or one or more pharmaceutically acceptable salts.

33. The Process according to claim 32 wherein the solution or suspension contains one or more amino acid(s) as excipient in addition to the low-molecular dextran and the pharmaceutically active substance.

34. The Process according to claim 32 wherein the solution or suspension contains isoleucine as excipient in addition to the low-molecular dextran and the pharmaceutically active substance.

35. The Process according to claim 32 wherein the solution or suspension contains a tripeptide as excipient in addition to the low-molecular dextran and the pharmaceutically active substance.

36. The Process according to claim 34 wherein the solution or suspension contains at least one isoleucine-containing tripeptide as excipient in addition to the low-molecular dextran and the pharmaceutically active substance.

37. The Process according to claim 36 wherein the solution or suspension contains triisoleucine as excipient in addition to the low-molecular dextran and the pharmaceutical active substance.

38. The Process according to claim 28 wherein the solution or suspension has a pH of between 3.0 and 9.0.

39. The Process according to claim 28 wherein amount of low-molecular dextran is between 50 and 99.99% (w/w) of the dry mass of the solution or suspension

40. The Process according to claim 39 wherein the amount of low-molecular dextran is between 60 and 99.99% (w/w) of the dry mass of the solution or suspension.

41. The Process according to claim 28 wherein the amount of pharmaceutically active substance is between 0.01 and 50% (w/w) of the dry mass of the solution or suspension.

42. The Process according to claims 28 wherein the dry mass of the solution or suspension contains at least 50% (w/w) of a low-molecular dextran and between 0.01 and 50% (w/w) of a biological macromolecule.

43. The Process according to claim 28 wherein the dry mass of the solution or suspension contains at least 60% (w/w) of a low-molecular dextran and between 0.01 and 40% (w/w) of a biological macromolecule.

44. The Process according to claims 28 wherein the dry mass of the solution or suspension contains at least 50% (w/w) of low-molecular dextran and between 1 and 20% (w/w) of at least one amino acid and/or at least one peptide.

45. The Process according to claim 44 wherein the dry mass of the solution or suspension contains at least 60% (w/w) of low-molecular dextran and between 1 and 20% (w/w) of at least one amino acid.

46. The Process according to claim 34 wherein the dry mass of the solution or suspension contains at least 60% (w/w) of low-molecular dextran and between 10 and 20% (w/w) of isoleucine.

47. The Process according to claim 38 wherein the dry mass of the solution or suspension contains at least 50% (w/w) of low-molecular dextran and between 1 and 20% (w/w) triisoleucine.

48. The Process according to claim 28 wherein the solution or suspension is sprayed to form a powder, the particles in the powder having an MMAD of between 1 and 10 μm.

49. The Process according to claim 48 wherein the solution or suspension is sprayed to form a powder, the particles in the powder having an MMAD of between 1 and 7.5 μm.

50. The process accrding to claim 28 wherein spraying is performed at a temperature below 150-185/80-95° C. (inflow/outflow temperature).
